(19) United States
(12) Patent Application Publication
(10) Pub. No.: US 2025/0011322 A1
(43) Pub. Date: Jan. 9, 2025

(54) MEDICAL COMPOUND FOR THE INHIBITION OF ALDEHYDE DEHYDROGENASE SUBTYPE 1A

(71) Applicant: Theranib Inc., Halifax (CA)

(72) Inventors: Concettina La Motta, Pisa (IT); Paola Marcato, Hubley (CA); Wasundara Fernando, Halifax (CA); Maya MacLean, Halifax (CA); Cheryl Dean, Halifax (CA); Giovanni Petrarolo, Matera (IT); Bianca Laura Bernardoni, Castiglioncello (IT); Ilaria D'Agostino, Sant'arcangelo (IT)

(21) Appl. No.: 18/757,309

(22) Filed: Jun. 27, 2024

(30) Foreign Application Priority Data
Jun. 29, 2023 (IT) 10202300013581

(51) Int. Cl. C07D 471/04; A61K 31/437; A61P 35/00

(52) U.S. Cl. CPC C07D 471/04; A61K 31/437; A61P 35/00

(57) ABSTRACT

Among other things, in general, compounds and pharmaceutical compositions thereof are disclosed involving small molecule inhibitors of ALDH1A isoforms. Medical compound for the inhibition of the activity of subtype 1A aldehyde dehydrogenase proteins is provided, including 2,6,8-trisubstituted imidazo[1,2-a]pyridines. Pathologies and diseases resulting from an upregulation of one or more ALDH1A isoforms may be prevented or treated thereby.

### CROSS-REFERENCE TO RELATED APPLICATIONS

This application claims the benefit of Italian patent application 10202300013581 entitled “Composto medicale per l'inibizione di aldeide deidrogenasi sottotipo 1A” filed Jun. 29, 2023, which is hereby incorporated by reference in its entirety.

### BACKGROUND

Aldehyde dehydrogenases are known to be NAD(P)+-dependent enzymes that catalyze the irreversible oxidation of endogenous and exogenous aldehydes to the corresponding carboxylic acids. They are widely expressed in the liver, kidney, heart, skeletal muscle, uterus, and brain, located in diverse cellular compartments including cytosol, mitochondria, endoplasmic reticulum, and nucleus.

The ALDH1A enzyme subfamily, including isoforms 1A1, 1A2, and 1A3 (hereafter collectively “ALDH1As”), specifically converts retinaldehyde into trans-retinoic acid, 9-cis retinoic acid, and 13-cis retinoic acid. These metabolites collectively regulate the transcription of more than 400 target genes associated with metabolism, immunity, organogenesis, and reproduction, thereby allowing ALDH1A enzymes to play a key role in multiple cellular processes. The alteration of the normal functioning of these enzymes therefore determines the onset of numerous pathologies such as cancer, obesity, type II diabetes, pulmonary arterial hypertension, and neointimal hyperplasia. Specifically, overexpression of specific ALDH1A isoforms has been associated with tumor growth and poor prognosis of several types of malignant tumors, both solid and non-solid, such as those of the head and neck, thyroid, lung, pleura, gastrointestinal, genitourinary, gynecological, breast, bone and soft tissue, skin, central nervous system, rare pediatric solids, lymphatic neoplasms, chronic and acute pediatric leukemias.

Furthermore, ALDH1As overexpression is also both a hallmark and vital element of a subset of cancer cells known as cancer stem cells (CSCs), which are responsible for tumor development and metastasis, as well as radio- and chemo-resistance. Therefore, the inhibition of the ALDH1A enzymes, including the isoforms 1A1, 1A2, and 1A3, represents a promising therapeutic approach for the treatment of cancer along with the selective eradication of CSCs.

ALDH1As activity is also important for the induction and function of Treg cells, mediated by the production of retinoic acid by multiple cell types such as dendritic cells, macrophages, and eosinophils. Thus, ALDH1As inhibition may increase the ratio of effector T cells to Treg cells within tumor tissue, leading to increased tumor immunity and tumor rejection.

ALDH1As promote the proliferation of vascular smooth muscle cells and pulmonary arteries. The resulting neointima formation narrows the luminal space, leading to pathologies such as neointimal hyperplasia, a major cause of restenosis, and pulmonary arterial hypertension. Therefore, ALDH1As inhibition may reduce intimal hyperplasia and, thus, be useful for the treatment of restenosis, coronary angioplasty, stenting and bypass surgery, and in patients after transplantation.

ALDH1As overexpression, found in patients with type II diabetes, identifies dedifferentiated pancreatic beta cells, characterized by impaired insulin secretion and mitochondrial function. The inhibition of these enzymes reactivates the differentiation of beta cells, thus representing a therapeutic strategy to reduce glycemia and increase insulin secretion in diabetic subjects.

Finally, since it has been demonstrated in animal models that the lack of the ALDH1A enzymes confers resistance to the development of diet-induced obesity, these isoenzymes also represent a possible target for the development of drugs for the treatment and/or prevention of obesity.

The literature describes numerous heterocyclic derivatives as inhibitors of ALDH1As. However, compounds described in the prior art show some important drawbacks. In particular, the described compounds show poor pharmacokinetic properties, including the short half-life and the lack of oral bioavailability, which limits their use to intravenous or intraperitoneal administration. Furthermore, they show off-target toxicity and/or lack of in vivo efficacy. To date, there are no selective inhibitors for the ALDH1A enzyme isoforms which have received the market approval as drugs or are under clinical development.

Therefore, there is a clear need for potent and selective ALDH1As inhibitors with good pharmacokinetic profile, which are suitable for oral dosing and have minimal or no toxicity.

### SUMMARY

Among other advantages that will be apparent upon review of the description, examples, claims, and Figures herein, compounds described herein and pharmaceutically relevant compositions comprising compounds described herein have the ability to inhibit ALDH1A1, ALDH1A2, or ALDH1A3, or are selective inhibitors of ALDH1A3. The compounds and compositions described herein have uses and methods of prevention and treatment of pathologies and diseases resulting from an upregulation of one or more ALDH1A isoforms, including but not limited to tumors and inflammatory diseases.

In general, in an aspect, a compound of Formula I, or a pharmaceutically acceptable salt or prodrug thereof, is described herein:

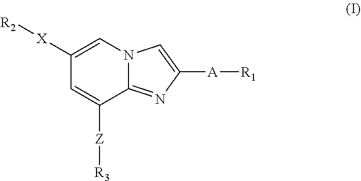

where A, X, and Z are each independently selected from aryl, heteroaryl, heterocyclic, or cyclic groups;
R₁ is selected from hydrogen, amino, fluoro, chloro, trifluoromethyl, cyano, nitro, carboxy, formyl, carbamoyl, acetyl, sulfamoyl, mercapto, acrylaldehyde, acrylate, acrylamide, acrylonitrile, dichloroacetamide, C1-C8 alkyl, C2-C8 alkenyl, C2-C8 alkynyl, C1-C6 alkoxy, C2-C6alkenyloxy, C2-C6alkynyloxy, C3-C7cycloalkyl, C3-C7heterocycloalkyl, hydroxyC1-C6alkyl, hydroxyC1-C6alkoxy, hydroxyC1-C6alkylamino, C1-C6alkoxyC1-C6alkyl, C1-C6alkoxyC1-C6alkylamino, aminoC1-C6alkyl, aminoC1-C6alkoxy, C1-C6alkylamino, di-C1-C6alkylamino, C1-C6-alkyldiamino, C1-C6alkoxycarbonyl, C1-C6alkoxyaminocarbonyl, C1-C6alkylthio, phosphateC1-C6alkylthio, sulfateC1-C6alkylthio, thioC1-C6alkyl, thioC1-C6alkoxy, thioC1-C6alkylamino, heterocycloC1-C6alkyl, heterocycloC1-C6alkoxy, heterocycloC1-C6alkylamino, phosphateC1-C6alkylamino, sulfateC1-C6alkylamino, C1-C6alkylsulfinyl, C1-C6alkylsulfonyl, C1-C6alkylthiocarboxamide, C1-C6alkylcarboxamide, C1-C6 alkylthioureyl, C1-C6alkylureyl, C1-C6dialkylamino, C1-C6alkoxycarbonyl, C1-C6dialkylcarboxamide, C1-C6 dialkylsulfonamide, C1-C6dialkylthiocarboxamido, C1-C6haloalkyl, C1-C6haloalkoxy, C1-C6guanidinealkyl, C1-C6cyanoalkyl, arylC1-C6alkoxy, heteroarylC1-C6alkoxy, cyclicC1-C6alkoxy, phosphateC1-C6alkoxy, sulfateC1-C6alkoxy, arylC1-C6alkylamino, heteroarylC1-C6alkylamino, cyclicC1-C6alkylamino, (((3R,4S,5S,6R)-3,4,5-trihydroxy-6-(hydroxymethyl)tetrahydro-2H-pyran-2-yl, aryl, heteroaryl, heterocyclic, and cyclic groups;
and R₂ and R₃ are each independently selected from hydrogen, halogen, fluoro, chloro, bromo, iodo, nitro, methyl, methoxy, cyano, formyl, carboxy, hydroxy, thiol, amino, amido, acetyl, sulfonamido, acryl, acrylaldehyde, but-3-en-2-one, methyl acrylate, N,N-dimethylacrylate, 2-(methylsulfonyl)vinyl, ethane-1-sulfonate, ethane-1-sulfonamido, acrylonitrile, nitrovinyl, pent-2-ene-1,4-dione, 4-oxobut-2-enoate, N,N-dimethyl-4-oxobut-2-enoate, ethan-1-one, and oxiran-2-yl-ethan-1-one, trifluoromethyl, carbamoyl, sulfamoyl, acrylamide, dichloroacetamide, C1-C8 alkyl, C2-C8 alkenyl, C2-C8 alkynyl, C1-C6 alkoxy, C2-C6alkenyloxy, C2-C6alkynyloxy, C3-C7cycloalkyl, C3-C7heterocycloalkyl, hydroxyC1-C6alkyl, hydroxyC1-C6alkoxy, hydroxyC1-C6alkylamino, C1-C6alkoxyC1-C6alkyl, C1-C6alkoxyC1-C6alkylamino, aminoC1-C6alkyl, aminoC1-C6alkoxy, C1-C6alkylamino, di-C1-C6alkylamino, C1-C6-alkyldiamino, C1-C6alkoxycarbonyl, C1-C6alkoxyaminocarbonyl, C1-C6alkylthio, phosphateC1-C6alkylthio, sulfateC1-C6alkylthio, thioC1-C6alkyl, thioC1-C6alkoxy, thioC1-C6alkylamino, heterocycloC1-C6alkyl, heterocycloC1-C6alkoxy, heterocycloC1-C6alkylamino, phosphateC1-C6alkylamino, sulfateC1-C6alkylamino, C1-C6alkylsulfinyl, C1-C6alkylsulfonyl, C1-C6alkylthiocarboxamide, C1-C6alkylcarboxamide, C1-C6 alkylthioureyl, C1-C6alkylureyl, C1-C6dialkylamino, C1-C6alkoxycarbonyl, C1-C6dialkylcarboxamide, C1-C6 dialkylsulfonamide, C1-C6dialkylthiocarboxamido, C1-C6haloalkyl, C1-C6haloalkoxy, C1-C6guanidinealkyl, C1-C6cyanoalkyl, arylC1-C6alkoxy, heteroarylC1-C6alkoxy, cyclicC1-C6alkoxy, phosphateC1-C6alkoxy, sulfateC1-C6alkoxy, arylC1-C6alkylamino, heteroarylC1-C6alkylamino, cyclicC1-C6alkylamino, (((3R,4S,5S,6R)-3,4,5-trihydroxy-6-(hydroxymethyl)tetrahydro-2H-pyran-2-yl, aryl, heteroaryl, heterocyclic, and cyclic groups;

And in which aryl, heteroaryl, heterocyclic, and cyclic groups may be further functionalized with additional substituents selected from hydrogen, halogen, fluoro, chloro, bromo, iodo, nitro, methyl, methoxy, cyano, formyl, carboxy, hydroxy, thiol, amino, amido, acetyl, sulfonamido, acryl, acrylaldehyde, but-3-en-2-one, methyl acrylate, N,N-dimethylacrylate, 2-(methylsulfonyl)vinyl, ethane-1-sulfonate, ethane-1-sulfonamido, acrylonitrile, nitrovinyl, pent-2-ene-1,4-dione, 4-oxobut-2-enoate, N,N-dimethyl-4-oxobut-2-enoate, ethan-1-one, and oxiran-2-yl-ethan-1-one, trifluoromethyl, carbamoyl, sulfamoyl, acrylamide, acrylonitrile, dichloroacetamide, C1-C6 alkyl, C2-C8 alkenyl, C2-C8 alkynyl, C1-C6 alkoxy, C2-C6 alkenyloxy, C2-C6 alkynyloxy, C1-C6 alkylcarboamino, C1-C6 alkylthio, C1-C6 alkylsulfonyl, C1-C6 alkylsulfonyl, C1-C6 alkylamino, di-C1-C6 alkylamino, amino C1-C6 alkyl, C3-C7 cycloalkyl, C1-C6 alkylcarbonyl, C2-C6 alkenylcarbonyl, C2-C6 alkynylcarbonyl, C1-C6 alkylaminocarbonyl, C1-C6 dialkyl aminocarbonyl, C1-C6 alkylsulfonyl aminocarbonyl, C1-C6 alkoxycarbonyl, arylaminocarbonyl and C3-C7 cycloalkylaminocarbonyl.

In some implementations, A, X, and Z are each phenyl; R₁ is selected from hydrogen, fluoro, chloro, bromo, iodo, nitro, methyl, methoxy, cyano, formyl, carboxy, thiol, amino, amido, acetyl, sulfonamido, acryl, acrylaldehyde, but-3-en-2-one, methyl acrylate, N,N-dimethylacrylate, 2-(methylsulfonyl)vinyl, ethane-1-sulfonate, ethane-1-sulfonamido, acrylonitrile, nitrovinyl, pent-2-ene-1,4-dione, 4-oxobut-2-enoate, N,N-dimethyl-4-oxobut-2-enoate, ethan-1-one, and oxiran-2-yl-ethan-1-one; and R₂ and R₃ are each independently selected from hydrogen, halogen, fluoro, chloro, bromo, iodo, nitro, methyl, methoxy, cyano, formyl, carboxy, hydroxy, thiol, amino, amido, acetyl, sulfonamido, acryl, acrylaldehyde, but-3-en-2-one, methyl acrylate, N,N-dimethylacrylate, 2-(methylsulfonyl)vinyl, ethane-1-sulfonate, ethane-1-sulfonamido, acrylonitrile, nitrovinyl, pent-2-ene-1,4-dione, 4-oxobut-2-enoate, N,N-dimethyl-4-oxobut-2-enoate, ethan-1-one, and oxiran-2-yl-ethan-1-one.

In some implementations, A, X, and Z are each phenyl; R₁ is selected from hydrogen, acetyl, formyl, alkyl, methyl, methyl ester (in either direction), methyl ether, fluoro, chloro, alkoxy, methoxy, and cyano; and R₂ and R₃ are each independently selected from hydrogen, acetyl, formyl, alkyl, methyl, methyl ester (in either direction), methyl ether, halogen, fluoro, chloro, hydroxy, alkoxy, methoxy, and cyano.

In some implementations, A, X, and Z are each phenyl; R₁ is selected from hydrogen, acetyl, formyl, alkyl, methyl, methyl ester (in either direction), methyl ether, fluoro, chloro, alkoxy, methoxy, and cyano; and R₂ and R₃ are each independently selected from hydrogen, acetyl, formyl, alkyl, methyl, methyl ester (in either direction), methyl ether, halogen, fluoro, chloro, hydroxy, alkoxy, methoxy, and cyano; and extant aryl, heteroaryl, heterocyclic, and cyclic groups are further functionalized with up to two additional substituents selected from hydrogen, acetyl, formyl, alkyl, methyl, methyl ester (in either direction), methyl ether, halogen, fluoro, chloro, hydroxy, alkoxy, methoxy, and cyano.

In some implementations, the compound is a 2,6,8-triarylimidazo[1,2-a]pyridine selected from those compounds named or exemplified herein.

In some implementations, the compound is compounded or formulated with a pharmaceutically acceptable carrier, optionally including a delivery system such as liposomal delivery.

In general, in an aspect, a method of manufacture of a 2,6,8-trisubstituted imidazo[1,2-a]pyridine is described herein, the method including reacting an alpha-aminopyridine directly connected to bromine and to iodine with a beta-carbonyl alkyl halide directly connected to a first functional group to produce an imidazo[1,2-a]pyridine nucleus directly connected to the first functional group, then reacting the product sequentially with a coupling agent (e.g., a boronic acid) directly connected to a second functional group and a coupling agent directly connected to a third functional group; resulting in the 2,6,8-trisubstituted imidazo[1,2-a]pyridine. In some implementations, the method of manufacture results in 1,1′-((2-phenylimidazo[1,2-a]pyridine-6,8-diyl)bis(4,1-phenylene))bis(ethan-1-one). In some implementations, the method of manufacture results in 4-(8-(4-acetylphenyl)-2-phenylimidazo[1,2-a]pyridin-6-yl)benzonitrile.

In general, in an aspect, methods of treatment or prevention of a pathology or disease resulting from abnormal regulation of subtype 1A aldehyde dehydrogenase activity in a subject suffering therefrom are described herein. Methods of treatment include administering a therapeutically effective amount of an inhibitor of aldehyde dehydrogenase proteins, while methods of prevention include administering a prophylactically effective amount of an inhibitor of aldehyde dehydrogenase proteins. In some implementations, the inhibitor of aldehyde dehydrogenase proteins is a compound described herein. In some implementations, the pathology or disease is cancer, e.g., tumors. In some implementations, the pathology or disease is inflammatory disease, e.g., lung inflammation. In some implementations, the pathology or disease is selected from diabetes, psoriasis, rheumatoid arthritis, acute nephropathies, chronic nephropathies, arterial restenosis, auto-immune diseases, acute infections, eye diseases resulting from alteration of the microcirculation, and endometriosis.

### BRIEF DESCRIPTION OF DRAWINGS

FIG. 1. Confirmation of ALDH1A3 levels in breast cancer cells by western blot. MDA-MB-231 has low levels of ALDH1A3 and ALDH1A3 is overexpressed in cells. MDA-MB-468 and HCC1806 have elevated ALDH1A3 levels, and ALDH1A3 is reduced by knockdown.

FIG. 2A. Blot showing MDA-MB-231 natively has low levels of ALDH1A3 and ALDH1A1 but can be induced to selectively overexpress either isoform.

FIG. 2B. The pan-ALDH inhibitor DEAB (100 uM) inhibits the Aldefluor assay activity of ALDH1A3 and ALDH1A1 (solid blue and red lines) in MDA-MB-231 cells overexpressing ALDH1A3 or ALDH1A1 respectively.

FIG. 2C. Compound 1 inhibits Aldefluor activity in MDA-MB-231 cells induced by ALDH1A3 overexpression. (top) The plot of count vs Aldefluor shows that at all plotted concentrations of compound 1, compound 1 inhibits Aldefluor activity in MDA-MB-231 cells induced by ALDH1A3 overexpression, substantially overlapping the curve of 100 uM DEAB control rather than the no-compound control condition (shaded area). (While it is not necessary to distinguish the colors in order to understand the Figure, for clarity the data series reach the y-axis reading of “47 count” from left-to-right in the following order: 10 uM comp. 1, 100 nM comp. 1, 1 uM comp. 1, 100 nM comp. 1, 100 uM DEAB, 10 nM comp. 1, 1 nM comp. 1.) (bottom) Quantitative bar graph presentation of Aldefluor activity in the presence of a dilution series of compound 1 as compared to negative and DEAB positive control conditions, normalized to 1.0 for the negative control “0” bar. Significance determined by one-way Anova, p-value <0.0001=****.

FIG. 2D. Compound 1 does not inhibit Aldefluor activity in MDA-MD-231 cells induced by ALDH1A1 overexpression. (top) The plot of count vs Aldefluor shows that at all plotted concentrations of compound 1, compound 1 overlaps the no-compound control condition (shaded area) rather than the 100 uM DEAB curve (red). (bottom) Quantitative bar graph presentation of Aldefluor activity in the presence of a dilution series of compound 1 as compared to negative and DEAB positive control conditions, normalized to 1.0 for the negative control “0” bar. Significance determined by one-way Anova, p-value <0.0001=****.

FIG. 2E. % Inhibition of Aldefluor by compound 1 in MDA-MB-231 cells with ALDH1A3 overexpression.

FIG. 3A. Nanomolar concentration of compound 2 inhibits Aldefluor activity in MDA-MD-231 cells induced by ALDH1A3 overexpression (n=4). Quantitative bar graph presentation of Aldefluor activity in the presence of a dilution series of compound 2 as compared to the negative control condition, normalized to 1.0 for the negative control “0” bar.

FIG. 3B. % Inhibition of Aldefluor by compound 2 in MDA-MB-231 cells with ALDH1A3 overexpression. Significance determined by one-way Anova, p-value <0.01=**, <0.0001=****.

FIG. 4A. Micromolar amounts of GA-11 inhibit ALDH1A3-induced Aldefluor activity in MDA-MB-231 cells with ALDH1A3 overexpression (n=3). (top) 100 uM amounts of DEAB and GA11 are distinguishable from control counts (shaded area) in the plot of Aldefluor count; all other concentration series shown are not distinguishable from control counts. (bottom) Quantitative bar graph presentation of Aldefluor activity in the presence of a dilution series of GA11 as compared to negative and DEAB positive control conditions, normalized to 1.0 for the negative control bar. Significance determined by one-way Anova, p-value <0.0001=****.

FIG. 4B. Micromolar amounts of NR6 inhibit ALDH1A3-induced Aldefluor activity in MDA-MB-231 cells with ALDH1A3 overexpression (n=3). (top) 100 uM amounts of DEAB and 100 uM and 10 uM amount of NR6 are distinguishable from control counts (shaded area) in the plot of Aldefluor count; all other concentration series shown are not distinguishable from control counts. (bottom) Quantitative bar graph presentation of Aldefluor activity in the presence of a dilution series of NR6 as compared to negative and DEAB positive control conditions, normalized to 1.0 for the negative control bar. Significance determined by one-way Anova, p-value <0.0001=****.

FIG. 4C. % Inhibition of Aldefluor by NR6 in MDA-MB-231 cells with ALDH1A3 overexpression. Significance determined by one-way Anova, p-value <0.01=**, <0.0001=****.

FIGS. 5A, 5B, 5C. Nanomolar concentrations of compound 1 specifically inhibit the expression of ALDH1A3 target genes in breast cancer cells. Expression of DHRS3, RARB, ELF3, and RARRES1 in MDA-MB-231 cells with ALDH1A3 overexpression (FIG. 5A), MDA-MB-468 cells (with ALDH1A3 shRNA knockdown as control, FIG. 5B), and HCC1806 (with ALDH1A3 shRNA knockdown as control, FIG. 5C) is determined 24 hours after treatment with 0, 1, 10, or 100 nM compound 1 by QPCR. Significance determined by one-way Anova, p-value <0.5=*, <0.01=**, <0.001=***, <0.0001=****.

FIGS. 6A, 6B. Expression of ALDH1A3 target genes, RARB, ELF3, and RARRES1, in control and ALDH1A3 overexpressing MDA-MB-231 cells (FIG. 6A), and in control and ALDH1A3 knockdown MDA-MB-468 cells (FIG. 6B), after 24 hours of treatment with compound 2.

FIG. 7. Body weight of mice injected with MDA-MB-231 cells on day 1 and treated with compound 1. Mice were treated as outlined as described in the Example below. No significant changes were observed, as can be seen by the substantial overlap of the data series. The arrow indicates that treatment with compound 1 started on day 15.

FIG. 8. MDA-MB-231 tumor volumes in mice treated with compound 1 according to the groups described in the Example below (OE=overexpression). The arrow indicates that treatment with compound 1 started on day 15.

FIG. 9. Final MDA-MB-231 tumor volumes of mice treated with compound 1. Significance determined by one-way Anova, p-value <0.5=*, <0.0001=****.

FIG. 10. Final MDA-MB-231 tumor weights (g) in mice treated with compound 1. Treatment started day 15 post cancer cell implantation and the experiment ended on day 41. Significance determined by one-way Anova, p-value <0.5=*, <0.001=***.

FIG. 11. Pharmacodynamic analysis. Compound 1 reduces ALDH1A3-inducible RARB expression in MDA-MB-231 tumors. Tumors were harvested on day 41 and treatment began on day 15. Significance determined by one-way Anova, p-value <0.5=*, <0.01=**, <0.0001=****.

FIG. 12A. Daily treatment of compound 1 does not affect serum levels of alanine aminotransferase (ALT) in mice. Start of administration of compound 1 and started on day 15 and the ALT was measured on day 41.

FIG. 12B. Daily treatment of compound 1 does not affect serum levels of creatinine in mice. Start of administration of compound 1 and started on day 15 and the creatinine was measured on day 41.

### DETAILED DESCRIPTION

In this document, the measures, values, shapes, and geometric references (such as perpendicularity and parallelism), when associated with words like “approximately” or other similar terms such as “almost”, “about”, or “substantially”, are to be understood as less than measurement errors or inaccuracies due to production and/or manufacturing errors and, above all, unless there is a slight deviation from the value, measure, shape, or geometric reference which it is associated with. For example, these terms, if associated with a value, preferably indicate a divergence of no more than 10% of the value itself. Furthermore, when used, terms such as “first”, “second”, “superior”, “inferior”, “higher”, “lower”, “principal”, “main” and “secondary” do not necessarily identify an order, relationship priority, or relative position, but they can simply be used to more clearly distinguish between different components. The measurements and data reported in this text are to be considered, unless otherwise indicated, as performed in an ICAO International Standard Atmosphere (ISO 2533:1975).

According to an embodiment, compounds usable in the prevention and treatment of pathologies resulting from an abnormal regulation of the activity of subtype 1A aldehyde dehydrogenase proteins are disclosed. In some preferred embodiments, the compounds are inhibitors of 1A1, 1A2, 1A3 isoforms or a combination thereof.

Compounds disclosed herein promote an inhibitory activity against aldehyde dehydrogenase proteins, in particular, subtype 1A (and preferably the isoforms 1A1, 1A2, 1A3, and combinations thereof). In some embodiments, compounds disclosed herein are used in the prevention and treatment of pathologies resulting from an abnormal regulation of the activity of subtype 1A aldehyde dehydrogenase proteins such as isoforms 1A1, 1A2, and 1A3.

According to an embodiment, 2,6,8-trisubstituted imidazo[1,2-a]pyridine derivatives, i.e., one or more substituted imidazo[1,2-a]pyridine derivatives, are disclosed herein, with substitutions at positions 2, 6, and 8 of the imidazopyridine nucleus.

According to an embodiment, pharmaceutical compositions are disclosed herein, comprising a compound described herein along with a pharmaceutically acceptable carrier. In some embodiments, the carrier comprises one or more excipients. In some embodiments, a compound described herein is formulated into a pharmaceutically acceptable dosage form such as a tablet, granule, powder, capsule, syrup, elixir, aqueous solution, aqueous suspension, oil solution, oil suspension, emulsion or microemulsion, to be used for oral, intramuscular, intravenous or subcutaneous administration or topical.

According to an embodiment, a compound of Formula I is disclosed:

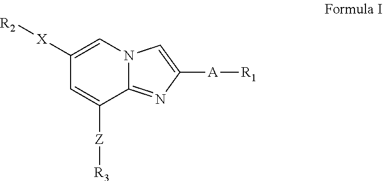

Where A, X, and Z are each independently selected from aryl, heteroaryl, heterocyclic, or cyclic groups;
R₁ is selected from hydrogen, amino, fluoro, chloro, trifluoromethyl, cyano, nitro, carboxy, formyl, carbamoyl, acetyl, sulfamoyl, mercapto, acrylaldehyde, acrylate, acrylamide, acrylonitrile, dichloroacetamide, C1-C8 alkyl, C2-C8 alkenyl, C2-C8 alkynyl, C1-C6 alkoxy, C2-C6alkenyloxy, C2-C6alkynyloxy, C3-C7cycloalkyl, C3-C7heterocycloalkyl, hydroxyC1-C6alkyl, hydroxyC1-C6alkoxy, hydroxyC1-C6alkylamino, C1-C6alkoxyC1-C6alkyl, C1-C6alkoxyC1-C6alkylamino, aminoC1-C6alkyl, aminoC1-C6alkoxy, C1-C6alkylamino, di-C1-C6alkylamino, C1-C6-alkyldiamino, C1-C6alkoxycarbonyl, C1-C6alkoxyaminocarbonyl, C1-C6alkylthio, phosphateC1-C6alkylthio, sulfateC1-C6alkylthio, thioC1-C6alkyl, thioC1-C6alkoxy, thioC1-C6alkylamino, heterocycloC1-C6alkyl, heterocycloC1-C6alkoxy, heterocycloC1-C6alkylamino, phosphateC1-C6alkylamino, sulfateC1-C6alkylamino, C1-C6alkylsulfinyl, C1-C6alkylsulfonyl, C1-C6alkylthiocarboxamide, C1-C6alkylcarboxamide, C1-C6 alkylthioureyl, C1-C6alkylureyl, C1-C6dialkylamino, C1-C6alkoxycarbonyl, C1-C6dialkylcarboxamide, C1-C6 dialkylsulfonamide, C1-C6dialkylthiocarboxamido, C1-C6haloalkyl, C1-C6haloalkoxy, C1-C6guanidinealkyl, C1-C6cyanoalkyl, arylC1-C6alkoxy, heteroarylC1-C6alkoxy, cyclicC1-C6alkoxy, phosphateC1-C6alkoxy, sulfateC1-C6alkoxy, arylC1-C6alkylamino, heteroarylC1-C6alkylamino, cyclicC1-C6alkylamino, (((3R,4S,5S,6R)-3,4,5-trihydroxy-6-(hydroxymethyl)tetrahydro-2H-pyran-2-yl, aryl, heteroaryl, heterocyclic, and cyclic groups; and
R₂ and R₃ are each independently selected from hydrogen, hydroxy, amino, halogen, trifluoromethyl, cyano, nitro, carboxy, formyl, carbamoyl, acetyl, sulfamoyl, mercapto, acrylaldehyde, acrylate, acrylamide, acrylonitrile, dichloroacetamide, C1-C8 alkyl, C2-C8 alkenyl, C2-C8 alkynyl, C1-C6 alkoxy, C2-C6alkenyloxy, C2-C6alkynyloxy, C3-C7cycloalkyl, C3-C7heterocycloalkyl, hydroxyC1-C6alkyl, hydroxyC1-C6alkoxy, hydroxyC1-C6alkylamino, C1-C6alkoxyC1-C6alkyl, C1-C6alkoxyC1-C6alkylamino, aminoC1-C6alkyl, aminoC1-C6alkoxy, C1-C6alkylamino, di-C1-C6alkylamino, C1-C6-alkyldiamino, C1-C6alkoxycarbonyl, C1-C6alkoxyaminocarbonyl, C1-C6alkylthio, phosphateC1-C6alkylthio, sulfateC1-C6alkylthio, thioC1-C6alkyl, thioC1-C6alkoxy, thioC1-C6alkylamino, heterocycloC1-C6alkyl, heterocycloC1-C6alkoxy, heterocycloC1-C6alkylamino, phosphateC1-C6alkylamino, sulfateC1-C6alkylamino, C1-C6alkylsulfinyl, C1-C6alkylsulfonyl, C1-C6alkylthiocarboxamide, C1-C6alkylcarboxamide, C1-C6 alkylthioureyl, C1-C6alkylureyl, C1-C6dialkylamino, C1-C6alkoxycarbonyl, C1-C6dialkylcarboxamide, C1-C6 dialkylsulfonamide, C1-C6dialkylthiocarboxamido, C1-C6haloalkyl, C1-C6haloalkoxy, C1-C6guanidinealkyl, C1-C6cyanoalkyl, arylC1-C6alkoxy, heteroarylC1-C6alkoxy, cyclicC1-C6alkoxy, phosphateC1-C6alkoxy, sulfateC1-C6alkoxy, arylC1-C6alkylamino, heteroarylC1-C6alkylamino, cyclicC1-C6alkylamino, (((3R,4S,5S,6R)-3,4,5-trihydroxy-6-(hydroxymethyl)tetrahydro-2H-pyran-2-yl, aryl, heteroaryl, heterocyclic, and cyclic groups.

In some embodiments, A, X, Z or combinations thereof may comprise monocyclic systems. In some embodiments, they may comprise bicyclic systems. In some heterocyclic embodiments of A, X, Z, or combinations thereof, one to three oxygen, nitrogen, phosphorus, or sulfur atoms are present. In some embodiments, A, X, and Z are different from each other. In some embodiments, A, X, and Z select the same moiety. In some embodiments, R₁, R₂, R₂ are different from each other. In some embodiments, R₁, R₂, R₃ select the same moiety. For clarity, in some embodiments any of R₁, R₂, or R₃ are all hydrogen and the corresponding A, X, or Z are therefore “functionalized with zero substituents”. In some preferred embodiments, R₁, R₂, R₃ are each independently attached to phenyl rings at the ortho, meta, or para positions, more preferably at para positions.

In some embodiments, aryl, heteroaryl, heterocyclic, and cyclic groups are further functionalized with one or more substituents selected from hydrogen, halogen, trifluoromethyl, cyano, nitro, amino, hydroxy, carboxy, formyl, carbamoyl, sulfamoyl, mercapto, acrylaldehyde, acrylate, acrylamide, acrylonitrile, dichloroacetamide, C1-C6 alkyl, C2-C8 alkenyl, C2-C8 alkynyl, C1-C6 alkoxy, C2-C6 alkenyloxy, C2-C6 alkynyloxy, C1-C6 alkylcarboamino, C1-C6 alkylthio, C1-C6 alkylsulfonyl, C1-C6 alkylsulfonyl, C1-C6 alkylamino, di-C1-C6 alkylamino, amino C1-C6 alkyl, C3-C7 cycloalkyl, C1-C6 alkylcarbonyl, C2-C6 alkenylcarbonyl, C2-C6 alkynylcarbonyl, C1-C6 alkylaminocarbonyl, C1-C6 dialkyl aminocarbonyl, C1-C6 alkylsulfonyl aminocarbonyl, C1-C6 alkoxycarbonyl, arylaminocarbonyl and C3-C7 cycloalkylaminocarbonyl. Said substituents can be the same or different from each other. In some embodiments, one or more R₁, R₂, R₃ substituents or one or more substituents or both are independently connected to A, X, Z up to the available positions on the aryl, heteroaryl, heterocyclic, or cyclic groups as applicable.

The terms “administration” or “administering” compound should be understood to mean providing a compound of the present invention to an individual in a form that can be introduced into that individual's body in an amount effective for prophylaxis, treatment, or diagnosis, as applicable. Such forms may include e.g., oral dosage forms, injectable dosage forms, transdermal dosage forms, inhalation dosage forms, and rectal dosage forms.

The term “alkyl” refers to a monovalent saturated hydrocarbon radical bearing a linear or branched residue, unless otherwise expressed. The “alkyl” group can contain double or triple carbon-carbon bonds when made up of two or more carbon atoms or can form cyclic residues when made up of at least three carbon atoms.

The term “alkenyl” as used herein means a straight chain, branched and/or cyclic hydrocarbon including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” as used herein means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkylamino” as used herein means an alkyl group, as defined herein, appended to the parent molecular moiety through a NH group. Representative examples of alkylamino include, but are not limited to, methylamino, ethylamino, isopropylamino, and butylamino.

The term “alkylcarbonyl” as used herein means an alkyl group, as defined herein, appended to the parent molecular moiety through a carbonyl group, as defined herein. Representative examples of alkylcarbonyl include, but are not limited to, methylcarbonyl, ethylcarbonyl, isopropylcarbonyl, n-propylcarbonyl, and the like.

The term “alkylsulfonyl” as used herein means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkynyl” as used herein means a straight or branched chain hydrocarbon group containing from 2 to 10 carbon atoms, and preferably 2, 3, 4, or 5 carbons, and containing at least one carbon-carbon triple bond. Representative examples of alkynyl include, but are not limited to, acetylenyl, 1-propynyl, 2-propynyl, 3-butynyl, 2-pentynyl, and 1-butynyl.

The term “amido” as used herein means an amino, alkylamino, or dialkylamino group appended to the parent molecular moiety through a carbonyl group, as defined herein. Representative examples of amido include, but are not limited to, aminocarbonyl, methylaminocarbonyl, dimethylaminocarbonyl, and ethylmethylaminocarbonyl.

The term “amino” as used herein means a —NH₂ group.

In this document, the term “aryl” refers to an aromatic monocyclic, bicyclic, or tricyclic carbon-loop system, consisting of carbon atoms between six and ten, for example, phenyl or naphthyl, unless otherwise expressed.

The term “arylalkyl” as used herein means an aryl group, as defined herein, appended to the parent molecular moiety through an alkyl group, as defined herein. Representative examples of arylalkyl include, but are not limited to, benzyl, 2-phenylethyl and 3-phenylpropyl.

The term “carbonyl” as used herein means a —C(═O)— group.

The term “carboxy” as used herein means a —COOH group, which may be protected as an ester group: —COO-alkyl.

By Cx, we mean herein an acyclic straight or branched hydrocarbon of longest length x; thus, C1-C5 includes methyl, ethyl, propyl, butyl, pentyl, isopropyl, etc.

The term “cyano” as used herein means a —CN group.

The term “cyanophenyl” as used herein means a —CN group appended to the parent molecular moiety through a phenyl group, including, but not limited to, 4-cyanophenyl, 3-cyanophenyl, and 2-cyanophenyl.

The term “cycle” or “cyclic” refers to a non-aromatic monocyclic, bicyclic, or tricyclic carbon-loop system containing from zero to three unsaturations and formed from a minimum of five to a maximum of twelve members.

The term “dialkylamino” as used herein means two independent alkyl groups, as defined herein, appended to the parent molecular moiety through a nitrogen atom. Representative examples of dialkylamino include, but are not limited to, dimethylamino, diethylamino, ethylmethylamino, and butylmethylamino.

The term “formyl” as used herein means a —C(O)H group.

The term “haloalkoxy” as used herein means at least one halogen, as defined herein, appended to the parent molecular moiety through an alkoxy group, as defined herein. Representative examples of haloalkoxy include, but are not limited to, 2-fluoroethoxy, trifluoromethoxy, and pentafluoroethoxy.

The term “halogen” refers to fluorine, chlorine, bromine, or iodine, unless otherwise indicated.

The term “heteroaryl” refers to a heteroaromatic monocyclic, bicyclic, or tricyclic carbon-loop system containing from zero to five unsaturations, formed from a minimum of five to a maximum of twelve members and containing from one to three heteroatoms, preferably selected from a group comprising N, O, S, P, SO, and SO₂. Monocyclic rings can include, but are not limited to, a six-membered aromatic ring wherein one to four of the ring carbon atoms are replaced by nitrogen atoms, five-membered rings containing a sulfur, oxygen, phosphorus or nitrogen in the ring; five-membered rings containing one to four nitrogen atoms; and five membered rings containing an oxygen, sulfur, or phosphorus and one to three nitrogen atoms. Representative examples of 5- to 6-membered monocyclic heteroaryl rings include, but are not limited to, furyl, imidazolyl, isoxazolyl, isothiazolyl, oxazolyl, pyrazinyl, pyrazolyl, pyridazinyl, pyridinyl, pyrimidinyl, pyrrolyl, tetrazolyl, [1,2,3]thiadiazolyl, [1,2,3]oxadiazolyl, thiazolyl, thienyl, [1,2,3]triazinyl, [1,2,4]triazinyl, [1,3,5]triazinyl, [1,2,3]triazolyl, and [1,2,4]triazolyl. Representative examples of bicyclic heteroaryl rings include, but are not limited to, indolyl, benzothienyl, benzofuranyl, indazolyl, benzimidazolyl, benzothiazolyl, benzoxazolyl, benzoisothiazolyl, benzoisoxazolyl, quinolinyl, isoquinolinyl, quinazolinyl, quinoxalinyl, phthalazinyl, pteridinyl, purinyl, naphthyridinyl, cinnolinyl, thieno[2,3-d]imidazole, thieno[3,2-b]pyridinyl, and pyrrolopyrimidinyl.

The term “heterocycle” or “heterocyclic” refers to a non-aromatic monocyclic, bicyclic, or tricyclic carbon-loop system containing from zero to three unsaturations, formed from a minimum of five to a maximum of twelve members and containing from zero to five unsaturations and from one to three heteroatoms, preferably selected from a group comprising N, O, S, P, SO, and SO₂. Functional groups can be the same or different from each other. Representative examples of nitrogen-containing heterocycles include, but are not limited to, azepanyl, azetidinyl, aziridinyl, azocanyl, dihydropyridazinyl, dihydropyridinyl, dihydropyrimidinyl, morpholinyl, piperazinyl, piperidinyl, pyrrolidinyl, pyrrolinyl, dihydrothiazolyl, dihydropyridinyl, and thiomorpholinyl. Representative examples of non-nitrogen containing non-aromatic heterocycles include, but are not limited to, dioxanyl, dithianyl, tetrahydrofuryl, dihydropyranyl, tetrahydropyranyl, and [1,3]dioxolanyl. Additional examples of heterocycles include, but are not limited to, azetidin-2-one, azepan-2-one, isoindolin-1,3-dione, (Z)-1H-benzo[e][1,4]diazepin-5(4H)-one, pyridazin-3(2H)-one, pyridin-2(1H)-one, pyrimidin-2(1H)-one, pyrimidin-2,4(1H,3H)-dione, pyrrolidin-2-one, benzo[d]thiazol-2(3H)-one, pyridin-4(1H)-one, imidazolidin-2-one, 1H-imidazol-2(3H)-one, piperidin-2-one, tetrahydropyrimidin-2(1H)-one, 1H-benzo[d]imidazol-2(3H)-one, [1,2,4]thiadiazolonyl, [1,2,5]thiadiazolonyl, [1,3,4]thiadiazinonyl, [1,2,4]oxadiazolonyl, [1,2,5]oxadiazolonyl, [1,3,4]oxadiazinonyl, and 1,5-dihydro-benzo[b][1,4]diazepin-2-on-yl.

The term “hydroxy” as used herein means an —OH group.

The term “hydroxyalkyl” as used herein means at least one hydroxy group, as defined herein, appended to the parent molecular moiety through an alkyl group, as defined herein. Representative examples of hydroxyalkyl include, but are not limited to, hydroxymethyl, 2-hydroxyethyl, 2-methyl-2-hydroxyethyl, 3-hydroxypropyl, 2,3-dihydroxypentyl, and 2-ethyl-4-hydroxyheptyl.

The term “imino” as defined herein means a —C(═NH)— group.

The term “mercapto” as used herein means a —SH group.

The term “nitro” as used herein means a —NO2 group.

The term “oxo” as used herein means (═O).

Unless otherwise indicated, the term “prodrug” encompasses pharmaceutically acceptable esters, carbonates, thiocarbonates, N-acyl derivatives, N-acyloxyalkyl derivatives, quaternary derivatives of tertiary amines, N-Mannich bases, Schiff bases, aminoacid conjugates, phosphate esters, metal salts and sulfonate esters of compounds disclosed herein. Examples of prodrugs include compounds that comprise a biohydrolyzable moiety (e.g., a biohydrolyzable amide, biohydrolyzable carbamate, biohydrolyzable carbonate, biohydrolyzable ester, biohydrolyzable phosphate, or biohydrolyzable ureide analog). Prodrugs of compounds disclosed herein are readily envisioned and prepared by those of ordinary skill in the art. See, e.g., Design of Prodrugs, Bundgaard, A. Ed., Elseview, 1985; Bundgaard, hours. “Design and Application of Prodrugs,” ATextbook of Drug Design and Development, Krosgaard-Larsen and hours. Bundgaard, Ed., 1991, Chapter 5, p. 113-191; and Bundgaard, hours. Advanced Drug Delivery Review, 1992, 8, 1-38.

The expression “racemic mixture” defines a set of the two R and S enantiomers in a 50:50 ratio.

The expression “scalemic mixture” identifies a set of the two enantiomers R and S in any reciprocal proportion. By way of non-limiting example, it can be a mixture enriched (for example at 70%, 80%, 90%, or 99%) in one of the two enantiomers.

The term “sulfonyl” as used herein means a —S(O)₂— group.

The term “thioalkoxy” as used herein means an alkyl group, as defined herein, appended to the parent molecular moiety through a sulfur atom. Representative examples of thioalkoxy include, but are no limited to, methylthio, ethylthio, and propylthio.

In some embodiments, a compound of Formula I may exist in an unsolvated form. In some embodiments, it may exist in a solvated forms with pharmaceutically acceptable solvents such as water, ethanol, and the like. In some embodiments, a compound of Formula I may exist in stereoisomeric forms, e.g., it may contain one or more asymmetric carbon atoms, and therefore can be in optically active forms, scalemic mixtures, or racemic mixtures. In some embodiments, imidazo[1,2-a]pyridine derivatives according to Formula I have one or more asymmetric carbon atoms and therefore can be in optically active forms, or scalemic mixtures or racemic mixtures.

Individual stereoisomers (enantiomers and diastereomers) and mixtures of these are included within the scope of the present invention. The present invention also covers the individual isomers of the compounds represented by Formula I as mixtures with isomers thereof in which one or more chiral centers are inverted. Likewise, in some embodiments, compounds disclosed herein may exist in tautomeric forms other than that shown in Formula I and these are also included within the scope of the present invention.

Synthesis of optically active compounds can be carried out by following methodologies commonly known in the literature, with reference to optically inactive compounds and syntheses described herein. For example, an optically active compound can be obtained both by using optically pure compounds as starting products and by optical resolution of scalemic or racemic forms.

In some embodiments, a pharmaceutical composition comprises a compound of Formula I, a stereoisomer, a prodrug, a mutual drug, a hybrid compound or a pharmaceutically acceptable salt thereof as defined herein, and a pharmaceutically acceptable carrier or delivery system; some preferred non-limiting examples of carriers or delivery systems are liposomes, lipid or protein vesicles, albumin, cyclodextrin, (gold) nanoparticles, polymers, and nanosponges.

In a further non-limiting embodiment, compounds of Formula I can be in the form of salts. Said salts can be derived from inorganic or organic acids, salification with amino acids, or suitable organic or inorganic bases if an acid function is present in said derivatives. The invention includes within its scope all possible stoichiometric and non-stoichiometric forms of the salts of the medical compounds of Formula I.

In some embodiments, compounds of Formula I can include all suitable isotopic variations. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes such as ²H, ³H, ¹³C, ¹⁴C, ¹⁵N, ¹⁷O, ¹⁸O, ³¹P, ³²P, ³⁵S, ¹⁸F, and ³⁶Cl, respectively. Certain isotopic variations of the invention, for example, those in which a radioactive isotope such as ³H or ¹⁴C is incorporated, are useful in drug and/or substrate tissue distribution studies. Further, substitution with isotopes such as deuterium ²H, may afford certain therapeutic advantages resulting from greater metabolic stability. Isotopic variations of the compounds of the invention can generally be prepared by conventional procedures such as by the illustrative methods or by the preparations described in the examples hereafter using appropriate isotopic variations of suitable reagents.

In some embodiments, pharmaceutical compositions comprise at least one compound of Formula I or a pharmaceutically acceptable salt or solvate thereof and one or more pharmaceutically acceptable carriers, excipients, adjuvants, and/or diluents. Pharmaceutically acceptable salt(s) are well-known in the art. For clarity, the term “pharmaceutically acceptable salts” as used herein generally refers to salts prepared from pharmaceutically acceptable non-toxic acids or bases including inorganic acids and bases and organic acids and bases. Suitable pharmaceutically acceptable base addition salts include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences, 18 th ed. (Mack Publishing, Easton Pa.: 1990) and Remington: The Science and Practice of Pharmacy, 19th ed. (Mack Publishing, Easton Pa.: 1995). The preparation and use of acid addition salts, carboxylate salts, amino acid addition salts, and zwitterion salts of compounds of the present invention may also be considered pharmaceutically acceptable if they are, within the scope of sound medical judgment, suitable for use in contact with the tissues of humans and lower animals without undue toxicity, irritation, allergic response, and the like, are commensurate with a reasonable benefit/risk ratio, and are effective for their intended use. Such salts may also include various solvates and hydrates of the compound of the present invention.

The term “excipient” or “pharmaceutically acceptable excipient”, as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

Unless otherwise indicated, the terms “prevent,” “preventing” and “prevention” contemplate an action that occurs before a patient begins to suffer from the specified disease or disorder, which inhibits or reduces the severity of the disease or disorder or of one or more of its symptoms. The terms encompass prophylaxis.

Unless otherwise indicated, a “prophylactically effective amount” of a compound is an amount sufficient to prevent a disease or condition, or one or more symptoms associated with the disease or condition, or prevent its recurrence. A prophylactically effective amount of a compound is an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, a “therapeutically effective amount” of a compound is an amount sufficient to treat a disease or condition, or one or more symptoms associated with the disease or condition.

The term “subject” is intended to include living organisms in which disease may occur. Examples of subjects include humans, monkeys, cows, sheep, goats, dogs, cats, mice, rats, and transgenic species thereof.

In some embodiments, a compound of Formula I is formulated as a pharmaceutical compositions chosen based on the treatment requirements. This may mean administration to a subject in need thereof in the form of tablets, capsules, oral preparations, powders, granules, pills, injectable, or infusible liquid solutions, suspensions, suppositories, syrup, elixir, aqueous solution, aqueous suspension, oil solution, oil suspension, emulsion, or microemulsion preparation for inhalation to be used for oral, intramuscular, intravenous, subcutaneous or topical administration.

The pharmaceutical compositions can be formulated for oral administration in solid or liquid form, for parenteral intravenous, subcutaneous, intramuscular, intraperitoneal, intraarterial, or intradermal injection, or for vaginal, nasal, topical, or rectal administration. Pharmaceutical compositions of the present invention suitable for oral administration can be presented as discrete dosage forms, e.g., tablets, chewable tablets, caplets, capsules, liquids, and flavored syrups. Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Parenteral dosage forms can be administered to patients by various routes including subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include solutions ready for injection, dry products ready to be dissolved or suspended in a pharmaceutically acceptable vehicle for injection, suspensions ready for injection, and emulsions. Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants. These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof. If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of materials which can be useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Dosage forms for topical administration may include powders, sprays, ointments and inhalants. A compound of the present invention can be mixed under sterile conditions with a pharmaceutically acceptable carrier and any needed preservatives, buffers or propellants which may be required. Ophthalmic formulations, eye ointments, powders and solutions are contemplated as being within the scope of this invention. Aqueous liquid compositions comprising compounds of the invention also are contemplated.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents. Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, ear drops, eye ointments, powders and solutions are also contemplated as being within the scope of this invention. The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together. Methods to form liposomes are known in the art. See, for example, Prescott, Ed., Methods in Cell Biology, Volume XIV, Academic Press, New York, N.Y., (1976), p 33 et seq.

Actual dosage levels of active ingredients in the pharmaceutical compositions of this invention can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

An effective amount of one of the compounds of the invention can be employed in pure form or, where such forms exist, in pharmaceutically acceptable salt form. Alternatively, the compound can be administered as a pharmaceutical composition containing the compound of interest in combination with one or more pharmaceutically acceptable carriers. It will be understood, however, that the total daily usage of the compounds and compositions of the invention will be decided by the attending physician within the scope of sound medical judgment. The specific effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; the risk/benefit ratio; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the compounds of the present invention as administered to a human or lower animal may range from about 0.0003 to about 30 mg/kg of body weight. For purposes of oral administration, more preferable doses can be in the range of from about 0.0003 to about 1 mg/kg body weight. If desired, the effective daily dose can be divided into multiple doses for purposes of administration; consequently, single dose compositions may contain such amounts or submultiples thereof to make up the daily dose. For oral administration, the compositions of the invention are preferably provided in the form of tablets containing about 1.0, about 5.0, about 10.0, about 15.0, about 25.0, about 50.0, about 100, about 250, or about 500 milligrams of the active ingredient.

In some embodiments, compounds of the present invention may be employed for use in the treatment and/or prevention of pathological conditions alone as a sole therapy or in combination with other therapeutic agents either by separate administrations or by including two or more active principles in the same pharmaceutical formulation. The components of the treatment may be administered simultaneously or sequentially or as a single dosage form containing both agents.

According to an embodiment, methods of therapy or prevention or uses of compounds of Formula I may also comprise administration of further antitumoral therapy, such as radiotherapy, immunotherapy, and chemotherapy. In some embodiments, the compounds of Formula I can be used in combination with or co-administrated with known anticancer agents or drugs; some preferred non-limiting examples are: mitomycin C, cisplatin, etoposide, vincristine, doxorubicin, isotretinoin, temozolomide, dasatinib, and cyclophosphamide.

Some preferred non-limiting examples of imidazo[1,2-a]pyridine derivatives of Formula I are reported below:
2,6,8-triphenylimidazo[1,2-a]pyridine
2-(4-chlorophenyl)-6,8-diphenylimidazo[1,2-a]pyridine
2-(3-chlorophenyl)-6,8-diphenylimidazo[1,2-a]pyridine
2-(2-chlorophenyl)-6,8-diphenylimidazo[1,2-a]pyridine
2-(4-bromophenyl)-6,8-diphenylimidazo[1,2-a]pyridine
2-(3-bromophenyl)-6,8-diphenylimidazo[1,2-a]pyridine
2-(2-bromophenyl)-6,8-diphenylimidazo[1,2-a]pyridine
2-(4-iodophenyl)-6,8-diphenylimidazo[1,2-a]pyridine
2-(3-iodophenyl)-6,8-diphenylimidazo[1,2-a]pyridine
2-(2-iodophenyl)-6,8-diphenylimidazo[1,2-a]pyridine
2-(4-nitrophenyl)-6,8-diphenylimidazo[1,2-a]pyridine
2-(3-nitrophenyl)-6,8-diphenylimidazo[1,2-a]pyridine
2-(2-nitrophenyl)-6,8-diphenylimidazo[1,2-a]pyridine
6,8-diphenyl-2-(p-tolyl)imidazo[1,2-a]pyridine
6,8-diphenyl-2-(m-tolyl)imidazo[1,2-a]pyridine
6,8-diphenyl-2-(o-tolyl)imidazo[1,2-a]pyridine
2-(4-methoxyphenyl)-6,8-diphenylimidazo[1,2-a]pyridine
2-(3-methoxyphenyl)-6,8-diphenylimidazo[1,2-a]pyridine
2-(2-methoxyphenyl)-6,8-diphenylimidazo[1,2-a]pyridine
4-(6,8-diphenylimidazo[1,2-a]pyridin-2-yl)benzonitrile
3-(6,8-diphenylimidazo[1,2-a]pyridin-2-yl)benzonitrile
2-(6,8-diphenylimidazo[1,2-a]pyridin-2-yl)benzonitrile
4-(6,8-diphenylimidazo[1,2-a]pyridin-2-yl)benzaldehyde
3-(6,8-diphenylimidazo[1,2-a]pyridin-2-yl)benzaldehyde
2-(6,8-diphenylimidazo[1,2-a]pyridin-2-yl)benzaldehyde
4-(6,8-diphenylimidazo[1,2-a]pyridin-2-yl)phenol
3-(6,8-diphenylimidazo[1,2-a]pyridin-2-yl)phenol
2-(6,8-diphenylimidazo[1,2-a]pyridin-2-yl)phenol
4-(6,8-diphenylimidazo[1,2-a]pyridin-2-yl)benzenethiol
3-(6,8-diphenylimidazo[1,2-a]pyridin-2-yl)benzenethiol
2-(6,8-diphenylimidazo[1,2-a]pyridin-2-yl)benzenethiol
4-(6,8-diphenylimidazo[1,2-a]pyridin-2-yl)aniline
3-(6,8-diphenylimidazo[1,2-a]pyridin-2-yl)aniline
2-(6,8-diphenylimidazo[1,2-a]pyridin-2-yl)aniline
4-(6,8-diphenylimidazo[1,2-a]pyridin-2-yl)benzamide
3-(6,8-diphenylimidazo[1,2-a]pyridin-2-yl)benzamide
2-(6,8-diphenylimidazo[1,2-a]pyridin-2-yl)benzamide
4-(6,8-diphenylimidazo[1,2-a]pyridin-2-yl)benzenesulfonamide
3-(6,8-diphenylimidazo[1,2-a]pyridin-2-yl)benzenesulfonamide
2-(6,8-diphenylimidazo[1,2-a]pyridin-2-yl)benzenesulfonamide
3-(4-(6,8-diphenylimidazo[1,2-a]pyridin-2-yl)phenyl)acrylaldehyde
4-(4-(6,8-diphenylimidazo[1,2-a]pyridin-2-yl)phenyl)but-3-en-2-one
methyl 3-(4-(6,8-diphenylimidazo[1,2-a]pyridin-2-yl)phenyl)acrylate
3-(4-(6,8-diphenylimidazo[1,2-a]pyridin-2-yl)phenyl)-N,N-dimethylacrylamide
2-(4-(2-(methylsulfonyl)vinyl)phenyl)-6,8-diphenylimidazo[1,2-a]pyridinemethyl
2-(4-(6,8-diphenylimidazo[1,2-a]pyridin-2-yl)phenyl)ethene-1-sulfonate
2-(4-(6,8-diphenylimidazo[1,2-a]pyridin-2-yl)phenyl)ethene-1-sulfonamide
3-(4-(6,8-diphenylimidazo[1,2-a]pyridin-2-yl)phenyl)acrylonitrile
2-(4-(2-nitrovinyl)phenyl)-6,8-diphenylimidazo[1,2-a]pyridine
1-(4-(6,8-diphenylimidazo[1,2-a]pyridin-2-yl)phenyl)pent-2-ene-1,4-dione
methyl 4-(4-(6,8-diphenylimidazo[1,2-a]pyridin-2-yl)phenyl)-4-oxobut-2-enoate
4-(4-(6,8-diphenylimidazo[1,2-a]pyridin-2-yl)phenyl)-N,N-dimethyl-4-oxobut-2-enamide
1-(3-(4-(6,8-diphenylimidazo[1,2-a]pyridin-2-yl)benzoyl)oxirane-2-yl)ethan-1-one
6-(4-chlorophenyl)-2,8-diphenylimidazo[1,2-a]pyridine
6-(3-chlorophenyl)-2,8-diphenylimidazo[1,2-a]pyridine
6-(2-chlorophenyl)-2,8-diphenylimidazo[1,2-a]pyridine
6-(4-bromophenyl)-2,8-diphenylimidazo[1,2-a]pyridine
6-(3-bromophenyl)-2,8-diphenylimidazo[1,2-a]pyridine
6-(2-bromophenyl)-2,8-diphenylimidazo[1,2-a]pyridine
6-(4-iodophenyl)-2,8-diphenylimidazo[1,2-a]pyridine
6-(3-iodophenyl)-2,8-diphenylimidazo[1,2-a]pyridine
6-(2-iodophenyl)-2,8-diphenylimidazo[1,2-a]pyridine
6-(4-nitrophenyl)-2,8-diphenylimidazo[1,2-a]pyridine
6-(3-nitrophenyl)-2,8-diphenylimidazo[1,2-a]pyridine
6-(2-nitrophenyl)-2,8-diphenylimidazo[1,2-a]pyridine
2,8-diphenyl-6-(p-tolyl)imidazo[1,2-a]pyridine
2,8-diphenyl-6-(m-tolyl)imidazo[1,2-a]pyridine
2,8-diphenyl-6-(o-tolyl)imidazo[1,2-a]pyridine
6-(4-methoxyphenyl)-2,8-diphenylimidazo[1,2-a]pyridine
6-(3-methoxyphenyl)-2,8-diphenylimidazo[1,2-a]pyridine
6-(2-methoxyphenyl)-2,8-diphenylimidazo[1,2-a]pyridine
4-(2,8-diphenylimidazo[1,2-a]pyridin-6-yl)benzonitrile
3-(2,8-diphenylimidazo[1,2-a]pyridin-6-yl)benzonitrile
2-(2,8-diphenylimidazo[1,2-a]pyridin-6-yl)benzonitrile
4-(2,8-diphenylimidazo[1,2-a]pyridin-6-yl)benzaldehyde
3-(2,8-diphenylimidazo[1,2-a]pyridin-6-yl)benzaldehyde
2-(2,8-diphenylimidazo[1,2-a]pyridin-6-yl)benzaldehyde
4-(2,8-diphenylimidazo[1,2-a]pyridin-6-yl)phenol
3-(2,8-diphenylimidazo[1,2-a]pyridin-6-yl)phenol
2-(2,8-diphenylimidazo[1,2-a]pyridin-6-yl)phenol
4-(2,8-diphenylimidazo[1,2-a]pyridin-6-yl)benzenethiol
3-(2,8-diphenylimidazo[1,2-a]pyridin-6-yl)benzenethiol
2-(2,8-diphenylimidazo[1,2-a]pyridin-6-yl)benzenethiol
4-(2,8-diphenylimidazo[1,2-a]pyridin-6-yl)aniline
3-(2,8-diphenylimidazo[1,2-a]pyridin-6-yl)aniline
2-(2,8-diphenylimidazo[1,2-a]pyridin-6-yl)aniline
4-(2,8-diphenylimidazo[1,2-a]pyridin-6-yl)benzamide
3-(2,8-diphenylimidazo[1,2-a]pyridin-6-yl)benzamide
2-(2,8-diphenylimidazo[1,2-a]pyridin-6-yl)benzamide
4-(2,8-diphenylimidazo[1,2-a]pyridin-6-yl)benzenesulfonamide
3-(2,8-diphenylimidazo[1,2-a]pyridin-6-yl)benzenesulfonamide
2-(2,8-diphenylimidazo[1,2-a]pyridin-6-yl)benzenesulfonamide
3-(4-(2,8-diphenylimidazo[1,2-a]pyridin-6-yl)phenyl)acrylaldehyde
4-(4-(2,8-diphenylimidazo[1,2-a]pyridin-6-yl)phenyl)but-3-en-2-one
methyl 3-(4-(2,8-diphenylimidazo[1,2-a]pyridin-6-yl)phenyl)acrylate
3-(4-(2,8-diphenylimidazo[1,2-a]pyridin-6-yl)phenyl)-N,N-dimethylacrylamide
6-(4-(2-(methylsulfonyl)vinyl)phenyl)-2,8-diphenylimidazo[1,2-a]pyridine
methyl 2-(4-(2,8-diphenylimidazo[1,2-a]pyridin-6-yl)phenyl)ethene-1-sulfonate
2-(4-(2,8-diphenylimidazo[1,2-a]pyridin-6-yl)phenyl)ethene-1-sulfonamide
3-(4-(2,8-diphenylimidazo[1,2-a]pyridin-6-yl)phenyl)acrylonitrile
6-(4-(2-nitrovinyl)phenyl)-2,8-diphenylimidazo[1,2-a]pyridine
1-(4-(2,8-diphenylimidazo[1,2-a]pyridin-6-yl)phenyl)pent-2-ene-1,4-dione
methyl 4-(4-(2,8-diphenylimidazo[1,2-a]pyridin-6-yl)phenyl)-4-oxobut-2-enoate
4-(4-(2,8-diphenylimidazo[1,2-a]pyridin-6-yl)phenyl)-N,N-dimethyl-4-oxobut-2-enamide
1-(3-(4-(2,8-diphenylimidazo[1,2-a]pyridin-6-yl)benzoyl)oxirane-2-yl)ethan-1-one
8-(4-chlorophenyl)-2,6-diphenylimidazo[1,2-a]pyridine
8-(3-chlorophenyl)-2,6-diphenylimidazo[1,2-a]pyridine
8-(2-chlorophenyl)-2,6-diphenylimidazo[1,2-a]pyridine
8-(4-bromophenyl)-2,6-diphenylimidazo[1,2-a]pyridine
8-(3-bromophenyl)-2,6-diphenylimidazo[1,2-a]pyridine
8-(2-bromophenyl)-2,6-diphenylimidazo[1,2-a]pyridine
8-(4-iodophenyl)-2,6-diphenylimidazo[1,2-a]pyridine
8-(3-iodophenyl)-2,6-diphenylimidazo[1,2-a]pyridine
8-(2-iodophenyl)-2,6-diphenylimidazo[1,2-a]pyridine
8-(4-nitrophenyl)-2,6-diphenylimidazo[1,2-a]pyridine
8-(3-nitrophenyl)-2,6-diphenylimidazo[1,2-a]pyridine
8-(2-nitrophenyl)-2,6-diphenylimidazo[1,2-a]pyridine
2,6-diphenyl-8-(p-tolyl)imidazo[1,2-a]pyridine
2,6-diphenyl-8-(m-tolyl)imidazo[1,2-a]pyridine
2,6-diphenyl-8-(o-tolyl)imidazo[1,2-a]pyridine
8-(4-methoxyphenyl)-2,6-diphenylimidazo[1,2-a]pyridine
8-(3-methoxyphenyl)-2,6-diphenylimidazo[1,2-a]pyridine
8-(2-methoxyphenyl)-2,6-diphenylimidazo[1,2-a]pyridine
4-(2,6-diphenylimidazo[1,2-a]pyridin-8-yl)benzonitrile
3-(2,6-diphenylimidazo[1,2-a]pyridin-8-yl)benzonitrile
2-(2,6-diphenylimidazo[1,2-a]pyridin-8-yl)benzonitrile
4-(2,6-diphenylimidazo[1,2-a]pyridin-8-yl)benzaldehyde
3-(2,6-diphenylimidazo[1,2-a]pyridin-8-yl)benzaldehyde
2-(2,6-diphenylimidazo[1,2-a]pyridin-8-yl)benzaldehyde
4-(2,6-diphenylimidazo[1,2-a]pyridin-8-yl)phenol
3-(2,6-diphenylimidazo[1,2-a]pyridin-8-yl)phenol
2-(2,6-diphenylimidazo[1,2-a]pyridin-8-yl)phenol
4-(2,6-diphenylimidazo[1,2-a]pyridin-8-yl)benzenethiol
3-(2,6-diphenylimidazo[1,2-a]pyridin-8-yl)benzenethiol
2-(2,6-diphenylimidazo[1,2-a]pyridin-8-yl)benzenethiol
4-(2,6-diphenylimidazo[1,2-a]pyridin-8-yl)aniline
3-(2,6-diphenylimidazo[1,2-a]pyridin-8-yl)aniline
2-(2,6-diphenylimidazo[1,2-a]pyridin-8-yl)aniline
4-(2,6-diphenylimidazo[1,2-a]pyridin-8-yl)benzamide
3-(2,6-diphenylimidazo[1,2-a]pyridin-8-yl)benzamide
2-(2,6-diphenylimidazo[1,2-a]pyridin-8-yl)benzamide
4-(2,6-diphenylimidazo[1,2-a]pyridin-8-yl)benzenesulfonamide
3-(2,6-diphenylimidazo[1,2-a]pyridin-8-yl)benzenesulfonamide
2-(2,6-diphenylimidazo[1,2-a]pyridin-8-yl)benzenesulfonamide
3-(4-(2,6-diphenylimidazo[1,2-a]pyridin-8-yl)phenyl)acrylaldehyde
4-(4-(2,6-diphenylimidazo[1,2-a]pyridin-8-yl)phenyl)but-3-en-2-one
methyl 3-(4-(2,6-diphenylimidazo[1,2-a]pyridin-8-yl)phenyl)acrylate
3-(4-(2,6-diphenylimidazo[1,2-a]pyridin-8-yl)phenyl)-N,N-dimethylacrylamide
8-(4-(2-(methylsulfonyl)vinyl)phenyl)-2,6-diphenylimidazo[1,2-a]pyridine
methyl 2-(4-(2,6-diphenylimidazo[1,2-a]pyridin-8-yl)phenyl)ethene-1-sulfonate
2-(4-(2,6-diphenylimidazo[1,2-a]pyridin-8-yl)phenyl)ethene-1-sulfonamide
3-(4-(2,6-diphenylimidazo[1,2-a]pyridin-8-yl)phenyl)acrylonitrile
8-(4-(2-nitrovinyl)phenyl)-2,6-diphenylimidazo[1,2-a]pyridine
1-(4-(2,6-diphenylimidazo[1,2-a]pyridin-8-yl)phenyl)pent-2-ene-1,4-dione
methyl 4-(4-(2,6-diphenylimidazo[1,2-a]pyridin-8-yl)phenyl)-4-oxobut-2-enoate
4-(4-(2,6-diphenylimidazo[1,2-a]pyridin-8-yl)phenyl)-N,N-dimethyl-4-oxobut-2-enamide
1-(3-(4-(2,6-diphenylimidazo[1,2-a]pyridin-8-yl)benzoyl)oxirane-2-yl)ethan-1-one
1,1′-((2-phenylimidazo[1,2-a]pyridine-6,8-diyl)bis(4,1-phenylene))bis(ethan-1-one)
4-(8-(4-acetylphenyl)-2-phenylimidazo[1,2-a]pyridin-6-yl)benzonitrile
4-(8-(4-acetylphenyl)-2-phenylimidazo[1,2-a]pyridin-6-yl)benzonitrile
1-(4-(6-(4-fluorophenyl)-2-phenylimidazo[1,2-a]pyridin-8-yl)phenyl)ethan-1-one
1-(4-(6-(4-chlorophenyl)-2-phenylimidazo[1,2-a]pyridin-8-yl)phenyl)ethan-1-one
1-(4-(6-(4-bromophenyl)-2-phenylimidazo[1,2-a]pyridin-8-yl)phenyl)ethan-1-one
1-(4-(6-(3,5-dimethoxyphenyl)-2-phenylimidazo[1,2-a]pyridin-8-yl)phenyl)ethan-1-one
dimethyl 4,4′-(2-phenylimidazo[1,2-a]pyridine-6,8-diyl)dibenzoate
methyl 4-(6-(4-cyanophenyl)-2-phenylimidazo[1,2-a]pyridin-8-yl)benzoate
methyl 4-(6-(3-cyanophenyl)-2-phenylimidazo[1,2-a]pyridin-8-yl)benzoate
methyl 4-(6-(4-fluorophenyl)-2-phenylimidazo[1,2-a]pyridin-8-yl)benzoate
methyl 4-(6-(4-chlorophenyl)-2-phenylimidazo[1,2-a]pyridin-8-yl)benzoate
methyl 4-(6-(4-bromophenyl)-2-phenylimidazo[1,2-a]pyridin-8-yl)benzoate
methyl 4-(6-(3,5-dimethoxyphenyl)-2-phenylimidazo[1,2-a]pyridin-8-yl)benzoate
6,8-bis(4-chlorophenyl)-2-phenylimidazo[1,2-a]pyridine
4-(8-(4-chlorophenyl)-2-phenylimidazo[1,2-a]pyridin-6-yl)benzonitrile
3-(8-(4-chlorophenyl)-2-phenylimidazo[1,2-a]pyridin-6-yl)benzonitrile
8-(4-chlorophenyl)-6-(4-fluorophenyl)-2-phenylimidazo[1,2-a]pyridine
6-(4-bromophenyl)-8-(4-chlorophenyl)-2-phenylimidazo[1,2-a]pyridine
8-(4-chlorophenyl)-6-(3,5-dimethoxyphenyl)-2-phenylimidazo[1,2-a]pyridine
1-(4-(8-(4-chlorophenyl)-2-phenylimidazo[1,2-a]pyridin-6-yl)phenyl)ethan-1-one
3-(8-(4-acetylphenyl)-2-(4-(2-hydroxyethoxy)phenyl)imidazo[1,2-a]pyridin-6-yl)benzonitrile
1-(4-(2-(4-((2-hydroxyethyl)amino)phenyl)-6-phenylimidazo[1,2-a]pyridin-8-yl)phenyl)ethan-1-one
3-(8-(4-acetylphenyl)-2-(4-((2-hydroxyethyl)amino)phenyl)imidazo[1,2-a]pyridin-6-yl)benzonitrile
1-(4-(6-(3,5-dimethoxyphenyl)-2-(4-((2-hydroxyethyl)amino)phenyl)imidazo[1,2-a]pyridin-8-yl)phenyl)ethan-1-one
3-(8-(4-acetylphenyl)-2-(4-((2-morpholinoethyl)amino)phenyl)imidazo[1,2-a]pyridin-6-yl)benzonitrile
3-(8-(4-acetylphenyl)-2-(4-((14-hydroxy-3,6,9,12-tetraoxatetradecyl)amino)phenyl)imidazo[1,2-a]pyridin-6-yl)benzonitrile3-(8-(4-acetylphenyl)-2-(4-((14-hydroxy-3,6,9,12-tetraoxatetradecyl)oxy)phenyl)imidazo[1,2-a]pyridin-6-yl)benzonitrile
1-(4-(6-(3,5-dimethoxyphenyl)-2-(4-((2-morpholinoethyl)amino)phenyl)imidazo[1,2-a]pyridin-8-yl)phenyl)ethan-1-one
1-(4-(6-(3,5-dimethoxyphenyl)-2-(4-((14-hydroxy-3,6,9,12-tetraoxatetradecyl)amino)phenyl)imidazo[1,2-a]pyridin-8-yl)phenyl)ethan-1-one1-(4-(6-(3,5-dimethoxyphenyl)-2-(4-((14-hydroxy-3,6,9,12-tetraoxatetradecyl)oxy)phenyl)imidazo[1,2-a]pyridin-8-yl)phenyl)ethan-1-one
3-(8-(4-acetylphenyl)-2-(4-(2-morpholinoethoxy)phenyl)imidazo[1,2-a]pyridin-6-yl)benzonitrile
1-(4-(6-(3,5-dimethoxyphenyl)-2-(4-(2-morpholinoethoxy)phenyl)imidazo[1,2-a]pyridin-8-yl)phenyl)ethan-1-one
1-(4-(2-(4-(2-(diethylamino)ethoxy)phenyl)-6-(3,5-dimethoxyphenyl)imidazo[1,2-a]pyridin-8-yl)phenyl)ethan-1-one
3-(8-(4-acetylphenyl)-2-(4-(2-(diethylamino)ethoxy)phenyl)imidazo[1,2-a]pyridin-6-yl)benzonitrile
1-(4-(6-(3,5-dimethoxyphenyl)-2-(4-((2-(piperazin-1-yl)ethyl)amino)phenyl)imidazo[1,2-a]pyridin-8-yl)phenyl)ethan-1-one
3-(8-(4-acetylphenyl)-2-(4-((2-(piperazin-1-yl)ethyl)amino)phenyl)imidazo[1,2-a]pyridin-6-yl)benzonitrile
3-(8-(4-acetylphenyl)-2-(4-(2-(piperazin-1-yl)ethoxy)phenyl)imidazo[1,2-a]pyridin-6-yl)benzonitrile
3-(8-(4-acetylphenyl)-2-(4-((2-(4-methylpiperazin-1-yl)ethyl)amino)phenyl)imidazo[1,2-a]pyridin-6-yl)benzonitrile
1-(4-(6-(3,5-dimethoxyphenyl)-2-(4-(2-(piperazin-1-yl)ethoxy)phenyl)imidazo[1,2-a]pyridin-8-yl)phenyl)ethan-1-one
1-(4-(6-(3,5-dimethoxyphenyl)-2-(4-((2-(4-methylpiperazin-1-yl)ethyl)amino)phenyl)imidazo[1,2-a]pyridin-8-yl)phenyl)ethan-1-one
N-(4-(8-(4-acetylphenyl)-6-(3,5-dimethoxyphenyl)imidazo[1,2-a]pyridin-2-yl)phenyl)-2-aminoacetamide
N-(4-(8-(4-acetylphenyl)-6-(3-cyanophenyl)imidazo[1,2-a]pyridin-2-yl)phenyl)-2-aminoacetamide
4-(8-(4-acetylphenyl)-6-(3-cyanophenyl)imidazo[1,2-a]pyridin-2-yl)phenyl acetate
4-(8-(4-acetylphenyl)-6-(3,5-dimethoxyphenyl)imidazo[1,2-a]pyridin-2-yl)phenyl acetate
1-(4-(6-(3,5-dimethoxyphenyl)-2-(4-(2-(((3R,4S,5S,6R)-3,4,5-trihydroxy-6-(hydroxymethyl)tetrahydro-2H-pyran-2-yl)amino)ethoxy)phenyl)imidazo[1,2-a]pyridin-8-yl)phenyl)ethan-1-one
3-(8-(4-acetylphenyl)-2-(4-(2-(((3R,4S,5S,6R)-3,4,5-trihydroxy-6-(hydroxymethyl)tetrahydro-2H-pyran-2-yl)amino)ethoxy)phenyl)imidazo[1,2-a]pyridin-6-yl)benzonitrile
1,1′-((2-phenylimidazo[1,2-a]pyridine-6,8-diyl)bis(4,1-phenylene))bis(ethan-1-one)
3-(8-(4-acetylphenyl)-2-phenylimidazo[1,2-a]pyridin-6-yl)benzonitrile
1-(4-(6-(4-fluorophenyl)-2-phenylimidazo[1,2-a]pyridin-8-yl)phenyl)ethane-1-one
1-(4-(6-(4-chlorophenyl)-2-phenylimidazo[1,2-a]pyridin-8-yl)phenyl)ethane-1-one
1-(4-(6-(3,5-dimethoxyphenyl)-2-phenylimidazo[1,2-a]pyridin-8-yl)phenyl)ethane-1-one
6,8-bis(4-chlorophenyl)-2-phenylimidazo[1,2-a]pyridine
2-phenyl-6,8-di-p-tolylimidazo[1,2-a]pyridine
6,8-bis(4-methoxyphenyl)-2-phenylimidazo[1,2-a]pyridine
4,4′-(2-phenylimidazo[1,2-a]pyridine-6,8-diyl)dibenzaldehyde
dimethyl 4,4′-(2-phenylimidazo[1,2-a]pyridine-6,8-diyl)dibenzoate
1,1′-((2-(4-hydroxyphenyl)imidazo[1,2-a]pyridine-6,8-diyl)bis(4,1-phenylene))bis(ethan-1-one)
1,1′-((2-(4-methoxyphenyl)imidazo[1,2-a]pyridine-6,8-diyl)bis(4,1-phenylene))bis(ethan-1-one)

According to an embodiment, synthetic methods are disclosed herein for the preparation of compounds and, in particular, the imidazo[1,2-a]pyridine derivatives of Formula I and of the corresponding salts, which can be used as drugs. More specifically, processes and methods for manufacturing the compounds are herein disclosed. In some embodiments, a process may initially comprise a cyclization step and at least one reaction step. In a cyclization step, a 2-substituted imidazo[1,2-a]pyridine derivative can be obtained, i.e. an imidazopyridine nucleus with the main group in position 2 and suitably a functional group R₁ connected to said group A (as those groups are defined according to Formula I herein). In such a cyclization step, 5-bromo-3-iodopyridin-2-amine (II) is cyclized, obtaining the corresponding 2-substituted imidazo[1,2-a]pyridine derivative (III) as depicted below using phenyl as the said functional group.

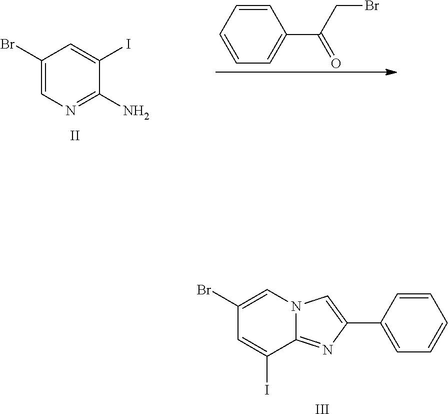

In some embodiments, for example, 5-bromo-3-iodopyridin-2-amine is reacted with one or more halides preferably having the following formula: R₁-A-C(O)CH₂—Halogen, where A and R₁ are selected as given herein for Formula I. The cyclization step can be carried out in the presence of a base catalyst, such as, for example, sodium or potassium carbonate. The process can comprise at least one reaction step in which at least one imidazo[1,2-a]pyridine derivative of Formula I is obtained. In this at least one reaction step, the imidazo[1,2-a]pyridine derivative of Formula I is obtained by reacting the 2-substituted imidazo[1,2-a]pyridine derivative III, suitably obtained in the cyclization step, with at least one substituted boronic acid; wherein in the whole of said at least reaction step the molar ratio between said 2-substituted imidazo[1,2-a]pyridine and said at least one boronic acid is 1:2. In such process, the at least one substituted boronic acid comprises at least one group selected from A and preferably at least one functional group selected from R₁ each R₁ being connected to only one A group. As a particular example, the at least one substituted boronic acid comprises only one A group and preferably only one R₁ functional group in molecular connection to the A group. As a more particular example, the acid may then be a phenylboronic acid. Preferably, in each of the at least one reaction step, the 2-substituted imidazo[1,2-a]pyridine derivative is reacted with only one of said boronic acid.

Therefore, according to an embodiment of the non-limiting process, the process comprises a single reaction step in which a 2-substituted imidazo[1,2-a]pyridine derivative is reacted with a single, substituted boronic acid, in ratio molar 1:2 to obtain a 2,6,8-trisubstituted imidazo[1,2-a]pyridine derivative. As an exemplary reaction step, a 2-substituted imidazo[1,2-a]pyridine derivative, Ill, is functionalized at positions 6 and 8 of the nucleus to give the corresponding 2,6,8-trisubstituted imidazo[1,2-a]pyridine derivative, IV as depicted below using phenyl as the said functional group.

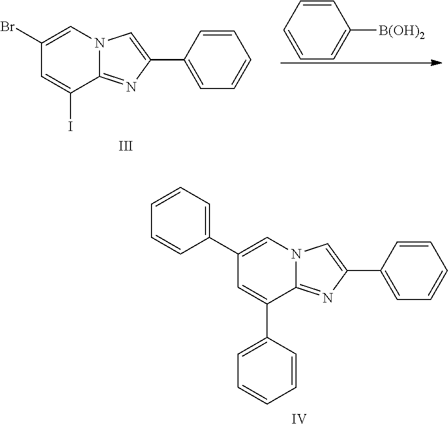

According to an embodiment of the non-limiting process, the process comprises a 2-substituted imidazo[1,2-a]pyridine derivative being reacted with a boronic acid preferably having the following formula: R₂—X—B(OH)₂ and R₃—Z—B(OH)₂, where X and Z are respectively selected from X and Z groups as given herein for Formula I; and R₂ and R₃ are selected as given herein for Formula I. In some embodiments, R₂ and R₃ select the same moiety. This single reaction step can be carried out in the presence of a catalyst, such as for example palladium acetate and triphenylphosphine, and a base, such as for example sodium carbonate.

In some embodiments, according to an embodiment of the non-limiting process, the process comprises at least two reaction steps. Specifically, in such embodiments, it comprises a first reaction step in which the 2,8-substituted imidazo[1,2-a]pyridine derivative V is obtained by reacting the 2-substituted imidazo[1,2-a]pyridine derivative with a first substituted boronic acid in molar ratio 1:1; and a second reaction step in which the 2,6,8-trisubstituted imidazo[1,2-a]pyridine derivative is obtained by reacting the 2,8-substituted imidazo[1,2-a]pyridine derivative with a second substituted boronic acid in a molar ratio of 1:1. The first substituted boronic acid and the second substituted boronic acid may be the same boronic acid or be different from each other. As an exemplary reaction step, the 2-substituted imidazo[1,2-a]pyridine derivative is reacted with an equimolar amount of a single, substituted boronic acid, so as to obtain the corresponding 2,8-substituted imidazo[1,2-a]pyridine derivative. As shown below as a non-limiting example, a 2-phenyl substituted imidazo[1,2-a]pyridine derivative, Ill, is functionalized in position 8 to give the corresponding 2,8-substituted imidazo[1,2-a]pyridine, V using phenyl as the said functional group.

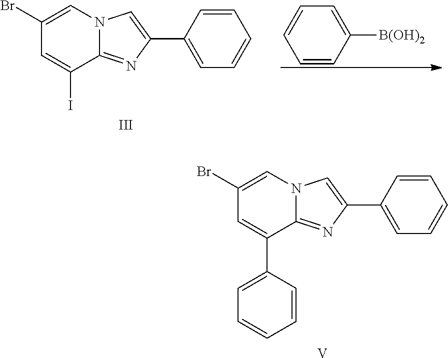

In some embodiments, the boronic acid used above is R₃—Z—B(OH)₂ where Z is selected from Z groups as given herein for Formula I; and R₃ is selected as given herein for Formula I. This reaction step can be carried out in the presence of a catalyst, such as for example palladium acetate and triphenylphosphine, and a base, such as for example sodium or potassium carbonate. Following this first reaction step according to this embodiment of the non-limiting process, a second reaction step is performed the 2,8-substituted imidazo[1,2-a]pyridine, V, is coupled to an equimolar amount of a single, substituted boronic acid, to obtain the corresponding 2,6,8-trisubstituted imidazo[1,2-a]pyridine. In this second reaction step, a 2,8-substituted imidazo[1,2-a]pyridine derivative, V, is functionalized at position 6 thus obtaining the 2,6,8-trisubstituted imidazo[1,2-a]pyridine derivative VI as depicted below using phenyl as the said functional group.

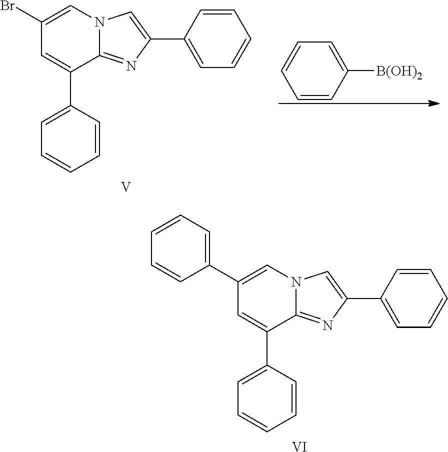

In some embodiments, the boronic acid used for the second reaction step given above is R₃—Z—B(OH)₂ where Z is selected from Z groups as given herein for Formula I; and R₃ is selected as given herein for Formula I. This second reaction step can be carried out in the presence of a catalyst, such as for example palladium acetate and triphenylphosphine or tetrakis(triphenylphosphine)palladium(0) and a base, such as sodium or potassium carbonate.

According to an embodiment, a method of manufacture is disclosed. The method comprises synthesis of a compound of Formula I comprising using any of the foregoing reaction steps. In a preferred embodiment, the method comprises using the following reaction scheme:

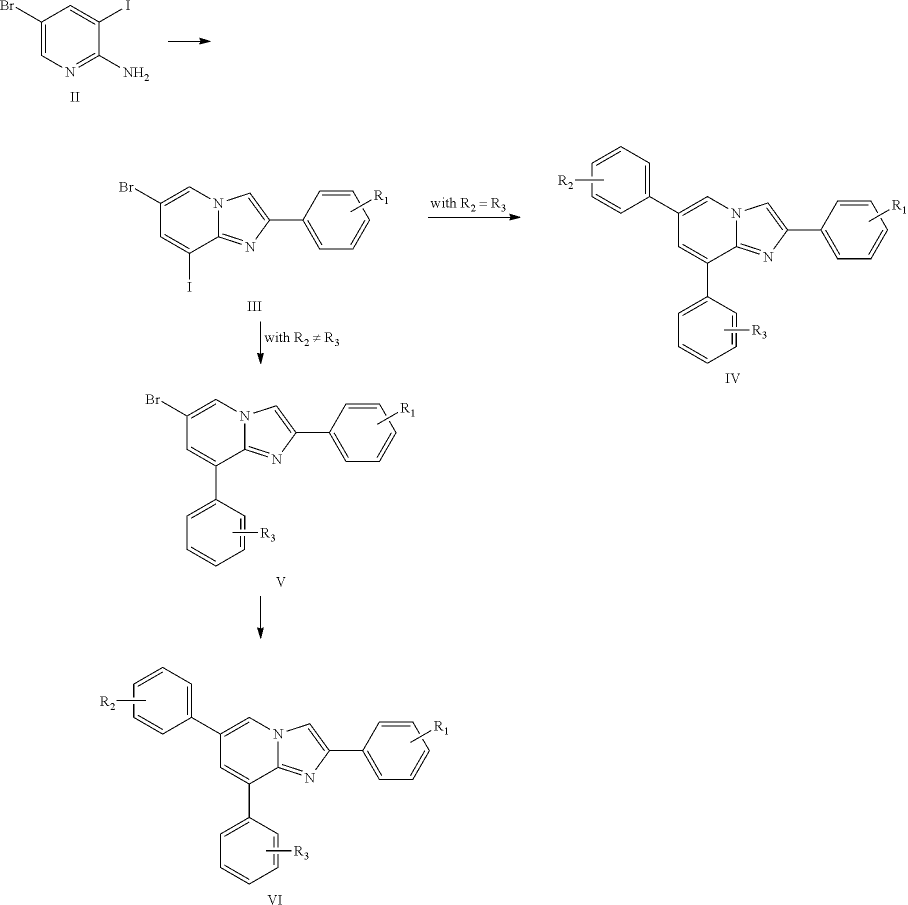

According to an embodiment, methods of treatment, methods of prevention, and uses of a compound of Formula I are disclosed. These comprise administering a compound of Formula I to a subject in need thereof for medical purposes, more specifically therapeutic or preventative purposes or both, where the subject is in need of an inhibitor of aldehyde dehydrogenase proteins; and, in particular, subtype 1A (and preferably of the isoforms 1A1, 1A2, and 1A3) for prevention, treatment, or both of pathologies and diseases resulting from abnormal regulation of the activity of the subtype 1A aldehyde dehydrogenase proteins such as the isoforms 1A1, 1A2, and 1A3.

In some embodiments, as a non-limiting example of medical utility of the inhibitory activity disclosed herein, a method of treatment and therefore the cure of tumors is disclosed, the method comprising administering a prophylactically or therapeutically effective amount (as applicable) of a compound of Formula I or at pharmaceutically acceptable salt thereof. In some embodiments, the tumor is solid. In some embodiments, the tumor is non-solid. In some embodiments, the tumor is a cancer selected from head and neck, thyroid, lung, pleura, gastrointestinal tract, genitourinary tract, gynecological, breast, bone and soft tissue, skin, central nervous system, neuroblastoma, glioblastoma, retinoblastoma, rhabdomyosarcoma, hepatocellular carcinoma, glioblastoma multiformae, melanoma, squamous cell carcinoma, pancreatic cancer, mesothelioma, and rare pediatric solid cancers. In some embodiments, the method is applicable in hemato-oncology in the treatment of lymphatic neoplasms, chronic leukemias, and acute leukemias of pediatric age.

In some embodiments, as a non-limiting example of medical utility of the inhibitory activity disclosed herein, a method of prevention or treatment or both of inflammatory diseases is disclosed, the method comprising administering prophylactically or therapeutically effective amount (as applicable) of a compound of Formula I. In some embodiments, the inflammatory disease impacts the respiratory tree, lungs, gastrointestinal tract, bile duct, the gallbladder, or combinations thereof.

In some embodiments, as a non-limiting example of medical utility of the inhibitory activity disclosed herein, a method of prevention or treatment or both of diseases of altered expression of subtype 1A aldehyde dehydrogenase proteins is disclosed, the method comprising administering a prophylactically or therapeutically effective amount (as applicable) of a compound of Formula I. In some embodiments, the disease of altered subtype 1A aldehyde dehydrogenase proteins is selected from diabetes, psoriasis, rheumatoid arthritis, acute nephropathies, chronic nephropathies, arterial restenosis, auto-immune diseases, acute infections, eye diseases resulting from alteration of the microcirculation, and endometriosis.

The invention is described hereinafter by means of experimental examples the purpose of which is simply to illustrate the invention in its operating details, but not to limit its scope of protection.

### EXAMPLES

### Example 1—Biological Data

The functional efficacy of the 2,6,8-trisubstituted imidazo[1,2-a]pyridine derivatives of Formula I has been experimentally verified by in vitro and in vivo assays.

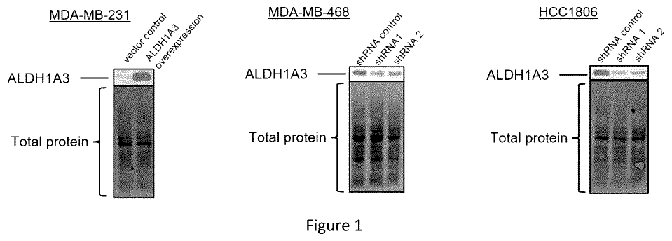

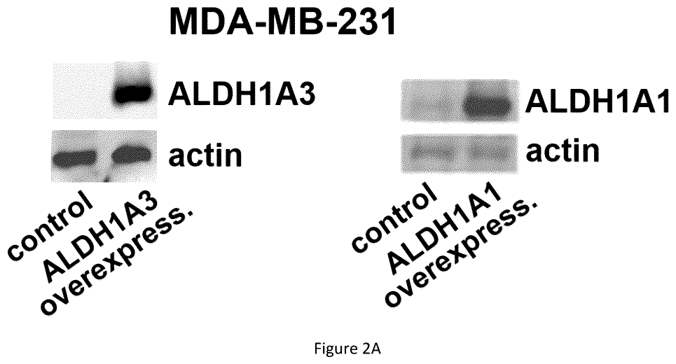

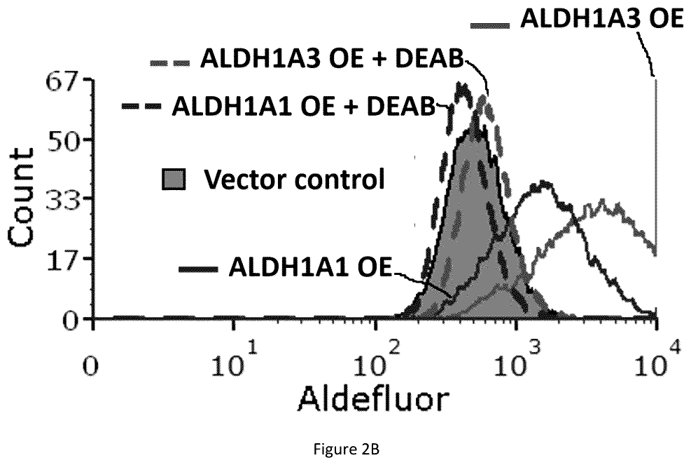

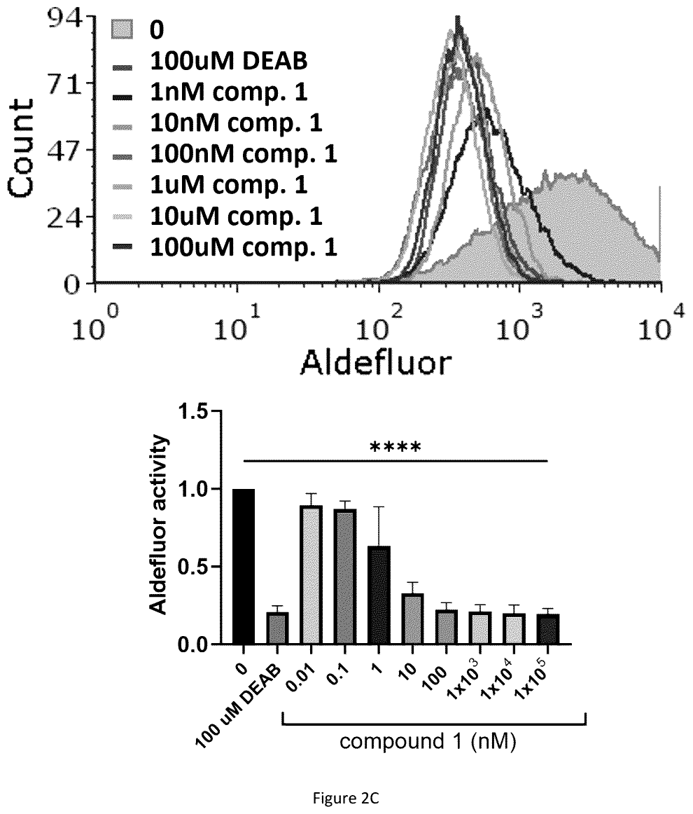

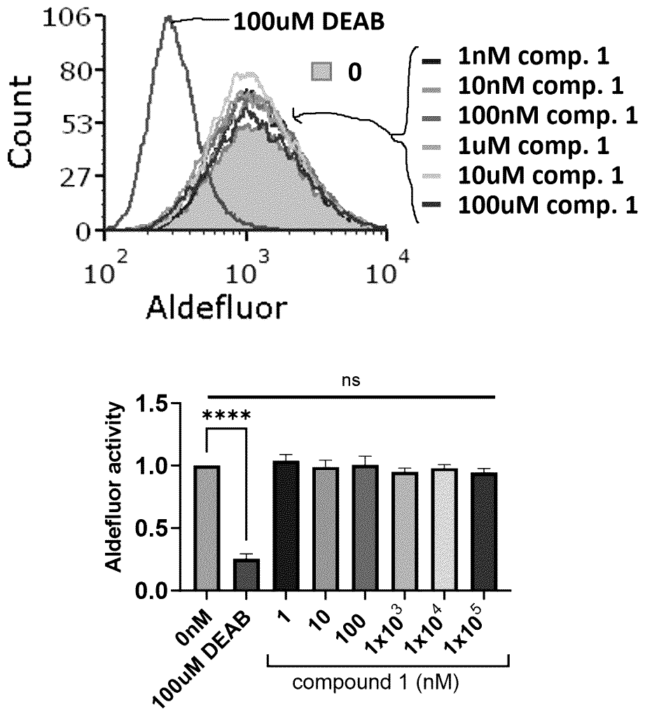

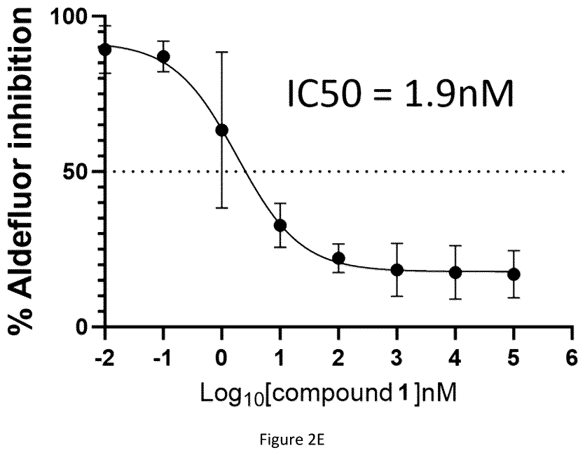

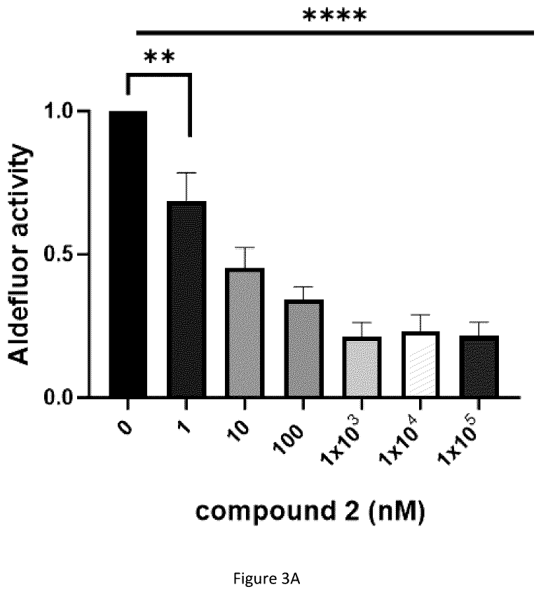

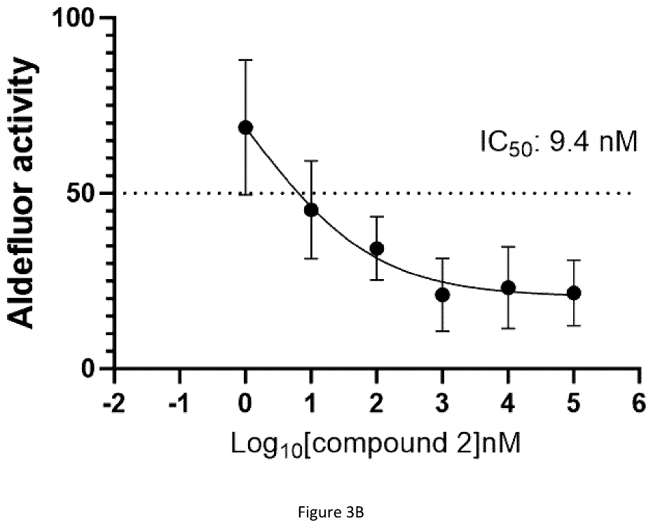

In vitro assays were conducted on breast cancer cell lines MDA-MB-231, MDA-MB-468, and HCC1806 cells. Specifically, the MDA-MB-231 cell line, characterized by a low expression of ALDH1A, was used as such or after being engineered to overexpress the ALDH1A1 or ALDH1A3 isoform, while the MDA-MB-468 and HCC1806 cell lines, characterized by a high expression of ALDH1A3 were used as such or after being knocked down for the ALDH1A3 isoform (FIG. 1). Initially, the ability of the compounds to specifically inhibit the activity of the ALDH1A3 versus ALDH1A1 isoform in MDA-MB-231 cells overexpressing the enzymes was determined (FIGS. 2A, 2B, 2C, 2D, 2E, 3A and 3B). The experiments were conducted using the Aldefluor® test, specific for aldehyde dehydrogenases.

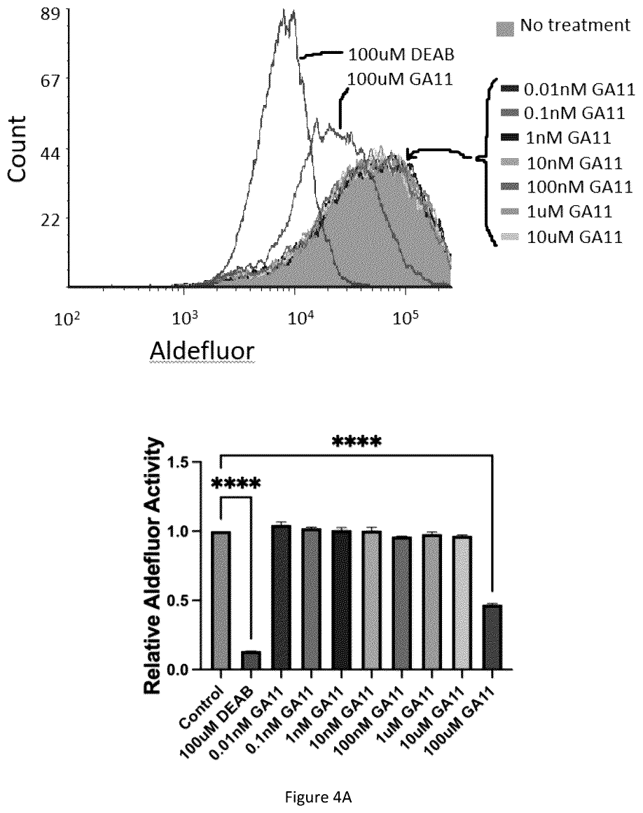

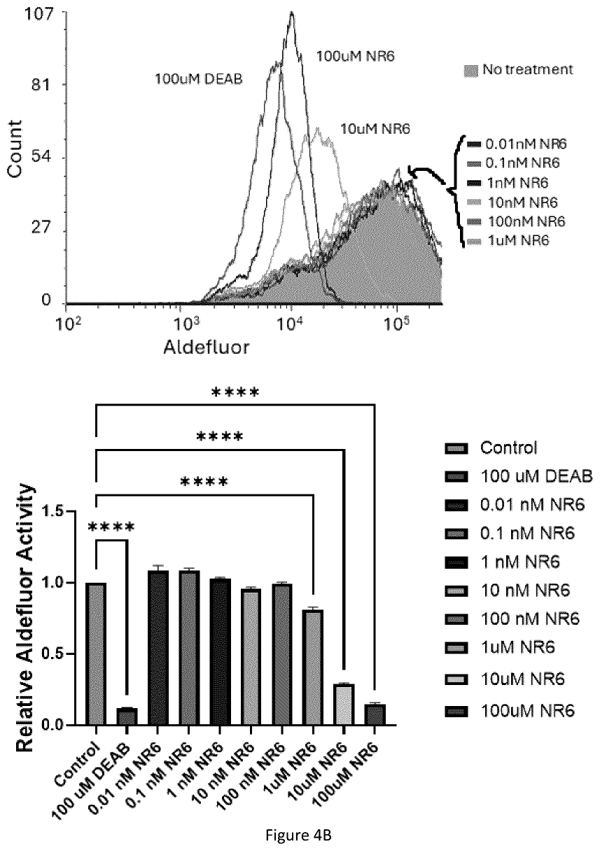

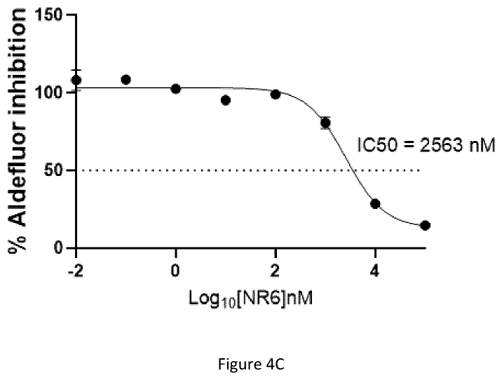

Table 1 summarizes the IC₅₀ data, the concentration producing 50% of the maximal effect of inhibition, obtained for compounds 1 and 2 given as examples (the synthesis for which is described below). In the experimental design, we also include two ALDH1A3 inhibitors, namely GA11 (reported as compound 3a in J. Med. Chem. 2020, 63, 9, 4603-4616, https://doi.org/10.1021/acs.jmedchem.9b01910) and NR6 (reported as compound 3h in J. Med. Chem. 2020, 63, 9, 4603-4616, https://doi.org/10.1021/acs.jmedchem.9b01910), for a direct comparison (FIGS. 4A, 4B, 4C). Compounds 1 and 2 show higher inhibitory activity against ALDH1A3 than do GA11 and NR6 and an activity that is selective for ALDH1A3:

TABLE 1

| Compound | IC₅₀ (nM) |
|---|---|
| 1 | 1.9 |
| 2 | 9.4 |
| GA11 | ~100 000 |
| NR6 | 2563 |

The efficacy of the test compounds was in Table 1 above compared to that of 4-Diethylaminobenzaldehyde (DEAB), a known inhibitor of ALDH1As used as a reference standard. Tested under the same experimental conditions, compounds 1 and 2 resulted in being active at a concentration that is over 1000 times lower than that used for DEAB, (FIGS. 2, 3, and 4). We confirmed that the activity of compound 1 is specific to ALDH1A3 and even a concentration of 100 uM of compound 1 did not inhibit the homologous enzyme ALDH1A1 (FIG. 2D). Concerning MDA-MB-468, no significant changes in the expression of the three target genes were observed in both the native and ALDH1A3 knockdown lines (FIGS. 3 and 4).

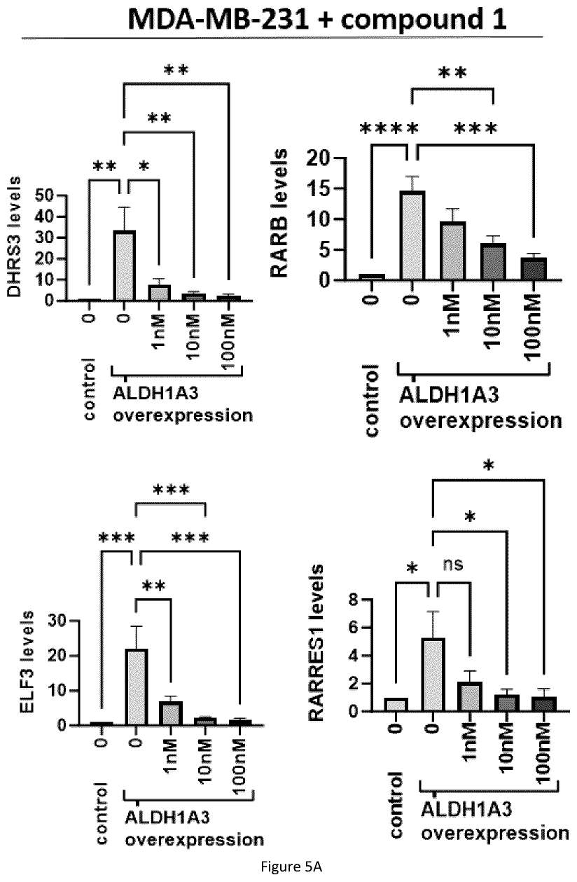

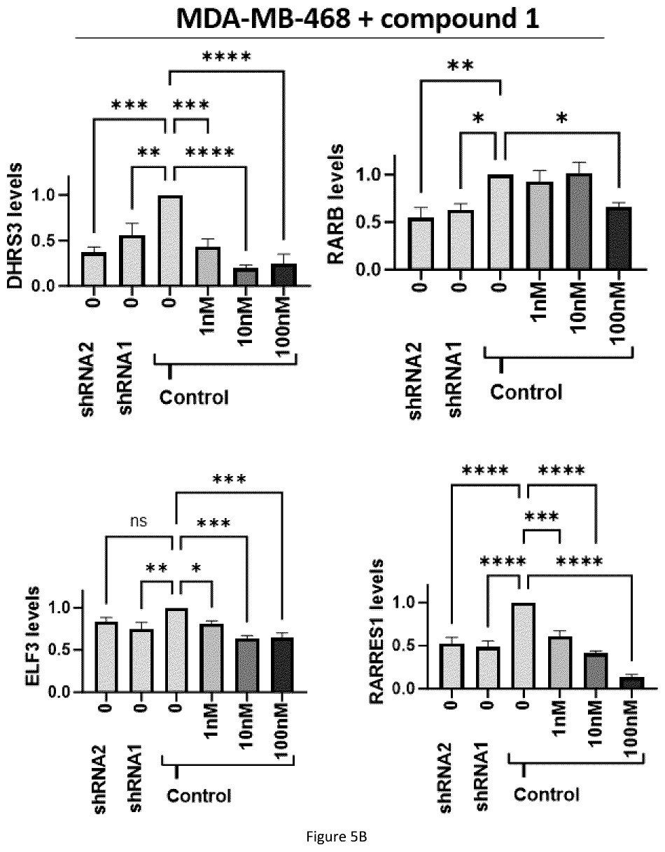

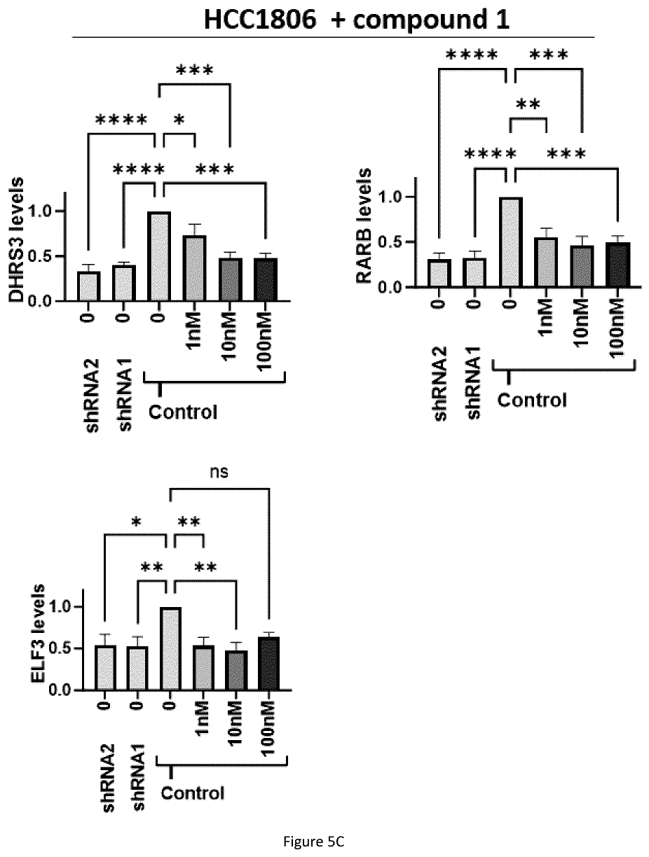

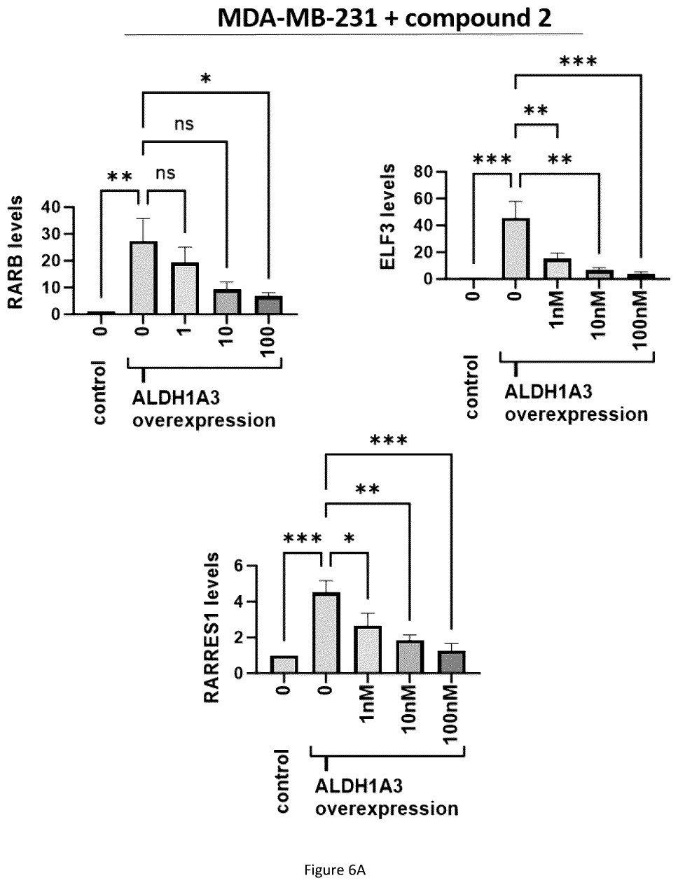

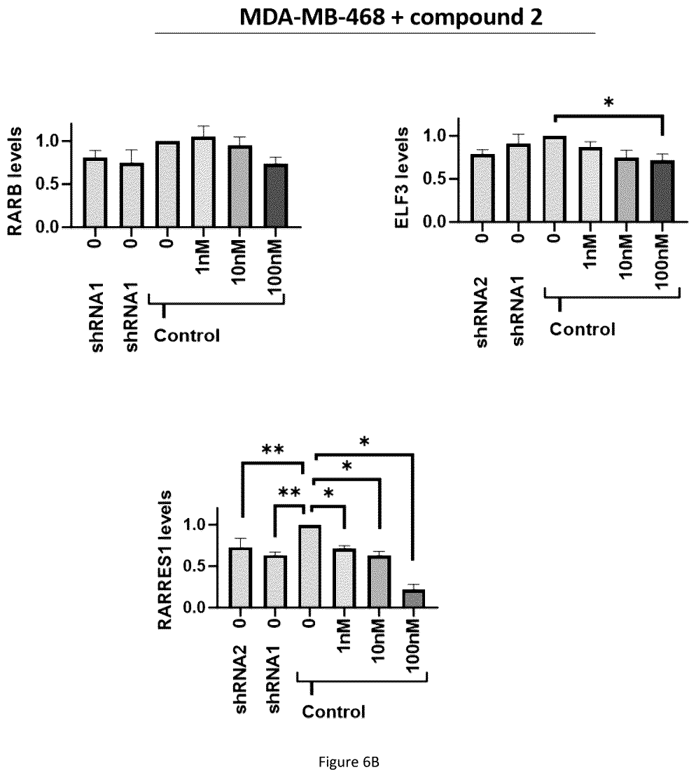

Subsequently, to confirm the direct inhibitory activity of the tested compounds on the enzymatic isoform of interest, their effect on the expression of ALDH1A3 target genes, and in particular of RARB, DHRS3, ELF3, and RARRES1, was investigated in the aforementioned cell lines. Regarding MDA-MB-231, after 24 hours of treatment, neither compound 1 nor compound 2 produced significant results in the native line but statistically significantly reduced the expression of the target genes in the overexpressing MDA-MB-231 cell line the enzyme and in MDA-MB-468 and HCC1806 cells (FIGS. 5A, 5B, 5C, 6A, and 6B).

In vivo experimentation then ensued. The in vivo assays were conducted using eight-week-old female NOD/SCID mice orthotopically injected with 2×106 native or ALDH1A3-overexpressing MDA-MB-231 cells. 60 female NOD-SCID mice had approximately 2,000,000 MDA-MB-231 cells injected into a 5^{th} mammary fat pad. 30 mice were injected with MDA-MB-231 bearing a vector control (Groups 1-3), and 30 mice were injected with MDA-MB-231 bearing an ALDH1A3 overexpression vector (Groups 4-6). On day 15 of inoculation, the treatment phase began. Groups 1 and 4 (n=10, 10 respectively) received saline i.p. injections 100 uL every day for 26 days. Groups 2 and 5 (n=10, 10) received 0.4 mg/kg drug i.p. in 100 uL saline every day for 26 days. Groups 3 and 6 (n=10, 10) received 4.0 mg/kg drug i.p. in 100 uL saline every day for 26 days. Caliper measurements of tumors and body weights were taken throughout the treatment phase. After 26 days, all mice were euthanized, the tumors were harvested, and blood was drawn for analysis.

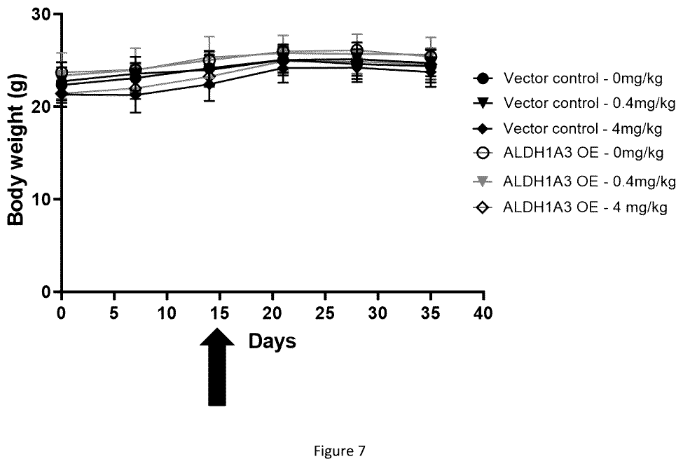

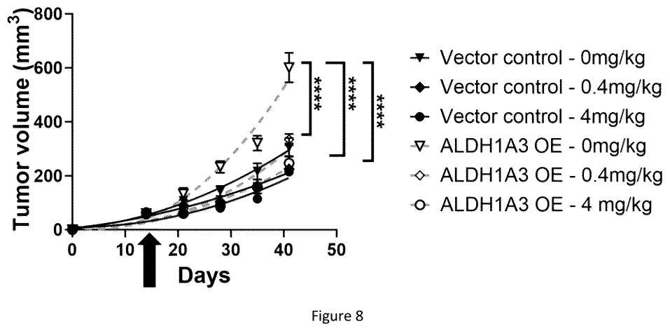

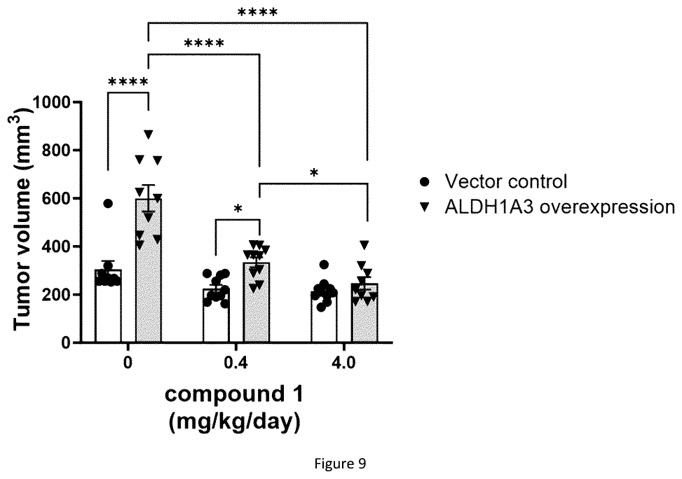

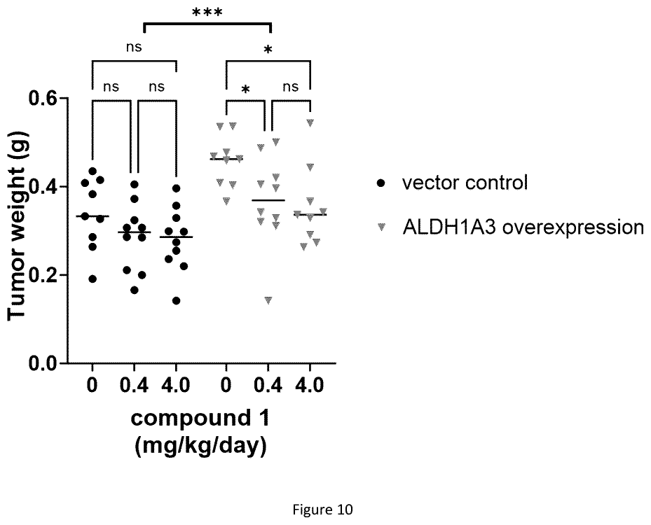

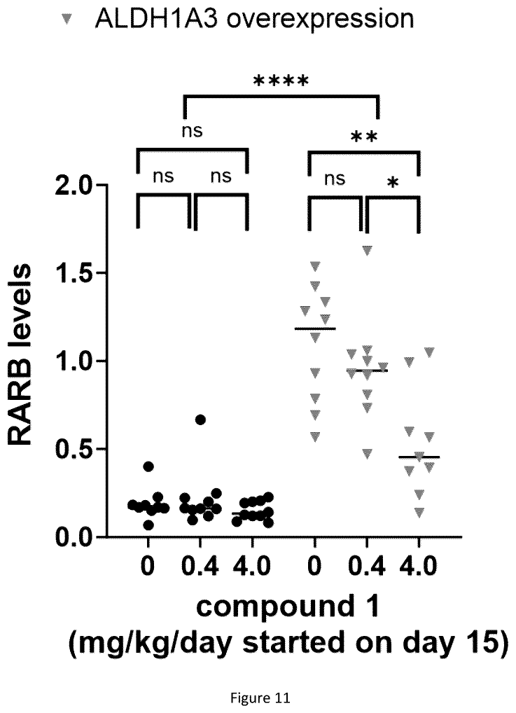

On day 15, the animals were treated with compound 1, administered daily intraperitoneally at a concentration of 0.4 mg/Kg or 4 mg/Kg, then constantly monitored for body weight increase (FIG. 7) and tumor volume (FIG. 8). Once the tumors became palpable, we started treatment of the animals (day 15). After 26 days of treatment, the animals were sacrificed and the tumors were collected and evaluated for size (FIGS. 8 and 9) and weight (FIG. 10). The test compound 1 has therefore been shown to reduce, in a statistically significant way, the increased tumor growth induced specifically by ALDH1A3 in xenotransplanted animals. Pharmacodynamic analysis of the collected tumors from sacrificed animals also demonstrates the specificity of the compound against ALDH1A3 in the tumors, as the animals treated with compound 1 had significantly reduced RARB level (induced by ALDH1A3 overexpression) in the tumors (FIG. 11).

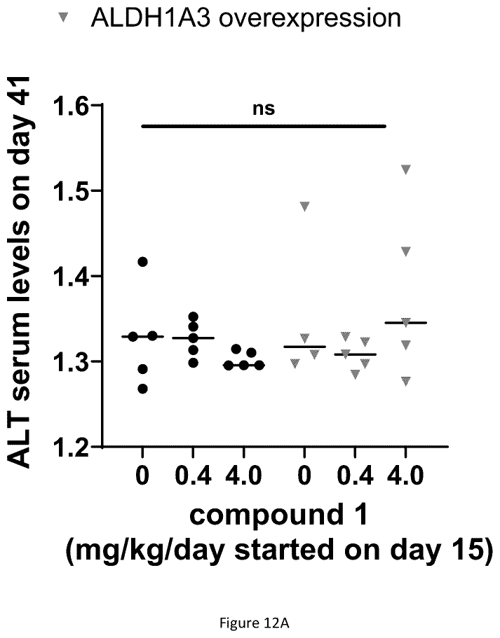

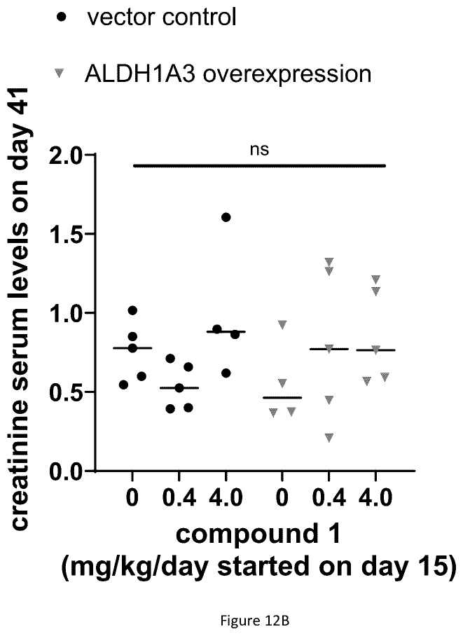

Importantly, there was no detectable toxicity induced by compound 1. This is evidenced by the trend over time of their body weight (FIG. 7) and also the analysis of alanine aminotransferase (ALT) and creatinine levels in serum collected from terminal animals (FIGS. 12A and 12B). The continued weight gain of the mice during treatment and the lack of changes in creatinine (increased creatinine is indicative of renal toxicity) and ALT levels (increased ALT is indicative of liver toxicity) suggest there is no toxicity associated with the daily treatment compound 1.

### Example 2—Synthetic Methods

Unless otherwise stated below, all the materials used, including the starting products, were obtained from commercial sources or prepared following experimental protocols described in the literature.

The information reported in the synthetic procedures described below refers to the following experimental conditions:
(i) temperatures are expressed in degrees Celsius (° C.);
(ii) the organic solutions were dried over anhydrous magnesium or sodium sulfate; the evaporation of the solvent was carried out using a rotary evaporator and working at reduced pressure;
(iii) thin layer chromatographies (TLC) were performed on Merck 60 F-254 plates; the column chromatographies were carried out using silica gel as the stationary phase in flash chromatography and/or using the Biotage Isolera Prime chromatographic system;
(iv) the time indicated in each procedure, necessary to obtain the desired product, was determined by TLC;
(v) the final product of each reaction was characterized by chemical-physical and spectroscopic data;
(vi) the yield reported for each product is indicative and does not necessarily correspond to that which can be obtained by means of an optimally carried out reaction;
(vii) 1H NMR spectra were recorded on a Bruker Ultrashield 400 spectrometer at 400 MHz. Spectra are reported in parts per million (6 scales) and internally referenced to the DMSO-d6 signal at δ 2.50 ppm. Data are reported as follows: chemical shifts, multiplicity (s=singlet, d=double, t=triplet, q=quartet, qi=quintet, m=multiple and/or multiplect resonance, bs=single broad), coupling constants (J) in Hertz (Hz) and integration.
(viii) purity of the final products (≥95%) was assessed by HPLC analysis, using a Shimadzu LC-20AD liquid chromatograph (PDA, 250-500 nm) and a Luna C18 column (250 mm×4.6 mm, 5 μm, Phenomenex), with an isocratic elution with a mixture of 30% water and 70% acetonitrile and a flow rate of 1.0 mL/min.

### Example 3—Synthesis of Compound 1: 1,1′-((2-phenylimidazo[1,2-a]pyridine-6,8-diyl)bis(4,1-phenylene))bis(ethan-1-one)

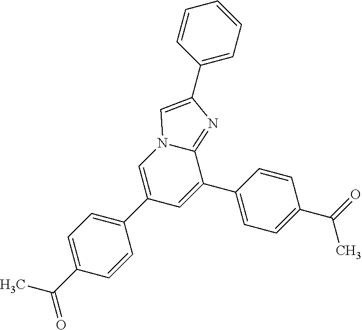 [SMILES: CC(=O)c1ccc(-c2cc(-c3ccc(C(C)=O)cc3)c3nc(-c4ccccc4)cn3c2)cc1]

A mixture of 2-amino-3-iodine-5-bromopyridine (1.00 mmol), 2-bromo-1-phenylethane-1-one (1.00 mmol) and sodium carbonate (1.00 mmol) in 20.0 mL of ethanol was refluxed under stirring until the disappearance of the starting material (TLC analysis). After cooling, the reaction mixture was evaporated to dryness and the resulting solid is purified by washing with water and subsequent crystallization from ethanol. The 2-phenyl-imidazo[1,2-a]pyridine thus obtained (1.00 mmol), suspended in ethanol/water (4:1), was then added to Pd(OAc)₂ (0.10 mmol), PPh₃ (0.20 mmol), 4-acetyl-phenylboronic acid (2.00 mmol) and sodium carbonate (2.00 mmol), and the resulting mixture was refluxed under stirring until the starting material disappears (TLC analysis). After cooling, the reaction mixture was evaporated to dryness under reduced pressure and the residue is purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 9.10 (d, J=1.7 Hz, 1H), 8.54 (d, J=2.4 Hz, 2H), 8.52 (d, J=1.8 Hz, 1H), 8.17-8.12 (m, 2H), 8.11 (d, J=8.5 Hz, 2H), 8.08-8.00 (m, 4H), 7.97 (d, J=1.8 Hz, 1H), 7.49 (t, J=7.7 Hz, 2H), 7.41-7.33 (m, 1H), 2.68 (s, 3H), 2.65 (s, 3H).

### Example 4—Synthesis of Compound 2: 4-(8-(4-acetylphenyl)-2-phenylimidazo[1,2-a]pyridin-6-yl)benzonitrile

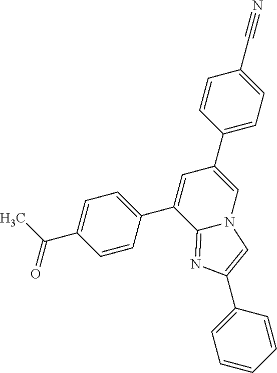 [SMILES: CC(=O)c1ccc(-c2cc(-c3ccc(C#N)cc3)cn3cc(-c4ccccc4)nc23)cc1]

A mixture of 2-amino-3-iodine-5-bromopyridine (1.00 mmol), 2-bromo-1-phenylethane-1-one (1.00 mmol) and Na₂CO₃ (1.00 mmol) in 20.0 mL of ethanol was refluxed under stirring until disappearance of the starting material (TLC analysis). After cooling, the reaction mixture was evaporated to dryness and the resulting solid is purified by washing with water and subsequent crystallization from ethanol. The 2-phenyl-imidazo[1,2-a]pyridine thus obtained (1.00 mmol) was then suspended in ethanol/water (4:1) and added to Pd(OAc)₂ (0.10 mmol), PPh₃ (0.20 mmol), 4-acetyl-phenylboronic acid (1.00 mmol) and Na₂CO₃ (2.00 mmol). The resulting mixture was refluxed under stirring until the disappearance of the starting material (TLC analysis). After cooling, it was evaporated to dryness under reduced pressure and the residue obtained is purified by column chromatography (silica gel, eluent mixture of ethyl acetate/petroleum ether). The 1-(4-(6-bromo-2-phenylimidazo[1,2-a]pyridin-8-yl)phenyl)ethane-1-one thus obtained (1.00 mmol) was then suspended in ethanol/water (4:1) and added with Pd(OAc)₂ (0.10 mmol), PPh₃ (0.20 mmol), 4-cyano-phenylboronic acid (1.00 mmol) and sodium carbonate (2.00 mmol). The resulting mixture was refluxed under stirring until the disappearance of the starting material (TLC analysis). After cooling, it was evaporated to dryness under reduced pressure and the residue obtained is purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 9.13 (d, J=1.8 Hz, 1H), 8.59-8.50 (m, 3H), 8.17-8.09 (m, 4H), 8.07-7.99 (m, 5H), 7.49 (t, J=7.7 Hz, 2H), 7.37 (t, J=7.6 Hz, 1H), 2.68 (s, 3H).

### Example 5—Synthesis of Compound 3: 3-(8-(4-acetylphenyl)-2-phenylimidazo[1,2-a]pyridin-6-yl)benzonitrile

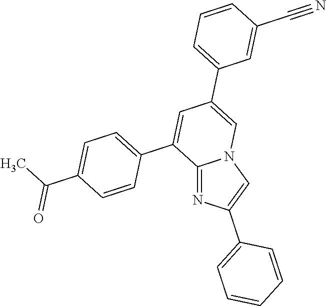 [SMILES: CC(=O)c1ccc(-c2cc(-c3cccc(C#N)c3)cn3cc(-c4ccccc4)nc23)cc1]

A mixture of 2-amino-3-iodine-5-bromopyridine (1.00 mmol), 2-bromo-1-phenylethane-1-one (1.00 mmol) and ₃ sodium carbonate (1.00 mmol) in 20.0 mL of ethanol was refluxed under stirring until the disappearance of the starting material (TLC analysis). After cooling, the reaction mixture was evaporated to dryness and the resulting solid is purified by washing with water and subsequent crystallization from ethanol. The 2-phenyl-imidazo[1,2-a]pyridine thus obtained (1.00 mmol) was then suspended in ethanol/water (4:1) and added to Pd(OAc)₂ (0.10 mmol), PPh₃ (0.20 mmol), 4-acetyl-phenylboronic acid (1.00 mmol) and sodium carbonate (2.00 mmol). The resulting mixture was refluxed under stirring until the disappearance of the starting material (TLC analysis). After cooling, it was evaporated to dryness under reduced pressure and the residue obtained is purified by column chromatography (silica gel, eluent mixture of ethyl acetate/petroleum ether). The 1-(4-(6-bromo-2-phenylimidazo[1,2-a]pyridin-8-yl)phenyl)ethane-1-one thus obtained (1.00 mmol) was then suspended in ethanol/water (4:1) and added with Pd(OAc)₂ (0.10 mmol), PPh₃ (0.20 mmol), 3-cyano-phenylboronic acid (1.00 mmol) and sodium carbonate (2.00 mmol). The resulting mixture was refluxed under stirring until the disappearance of the starting material (TLC analysis). After cooling, it was evaporated to dryness under reduced pressure and the residue obtained is purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 9.11 (d, J=1.8 Hz, 1H), 8.59-8.53 (m, 2H), 8.52 (s, 1H), 8.44 (t, J=1.8 Hz, 1H), 8.23 (m, 1H), 8.17-8.11 (m, 2H), 8.09-8.03 (m, 2H), 8.02 (d, J=1.8 Hz, 1H), 7.90 (m, 1H), 7.75 (t, J=7.8 Hz, 1H), 7.49 (t, J=7.7 Hz, 2H), 7.40-7.33 (m, 1H), 2.68 (s, 3H).

### Example 6—Synthesis of Compound 4: 1-(4-(6-(4-fluorophenyl)-2-phenylimidazo[1,2-a]pyridin-8-yl)phenyl)ethane-1-one

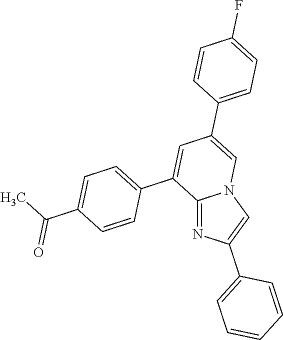 [SMILES: CC(=O)c1ccc(-c2cc(-c3ccc(F)cc3)cn3cc(-c4ccccc4)nc23)cc1]

A mixture of 2-amino-3-iodine-5-bromopyridine (1.00 mmol), 2-bromo-1-phenylethane-1-one (1.00 mmol) and sodium carbonate (1.00 mmol), in 20.0 mL of ethanol, was refluxed under stirring until the disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol) was then suspended in ethanol/water (4:1) and added with Pd(OAc)₂ (0.10 mmol), PPh₃ (0.20 mmol), 4-acetyl-phenylboronic acid (1.00 mmol) and sodium carbonate (2.00 mmol). The resulting mixture was refluxed under stirring until the disappearance of the starting material (TLC analysis). After cooling, it was evaporated to dryness under reduced pressure and the residue obtained was purified by column chromatography (silica gel, eluent mixture of ethyl acetate/petroleum ether). The 1-(4-(6-bromo-2-phenylimidazo[1,2-a]pyridin-8-yl)phenyl)ethane-1-one thus obtained (1.00 mmol) was then suspended in ethanol/water (4:1) and added with Pd(OAc)₂ (0.10 mmol), PPh₃ (0.20 mmol), 4-fluoro-phenylboronic acid (1.00 mmol) and sodium carbonate (2.00 mmol). The resulting mixture was refluxed under stirring until the disappearance of the starting material (TLC analysis). After cooling, it was evaporated to dryness under reduced pressure and the residue obtained was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 8.95 (d, J=1.7 Hz, 1H), 8.53 (d, J=1.7 Hz, 1H), 8.51 (d, J=1.3 Hz, 2H), 8.18-8.09 (m, 2H), 8.08-8.01 (m, 2H), 7.94-7.87 (m, 3H), 7.48 (t, J=7.6 Hz, 2H), 7.38 (t, J=8.6 Hz, 3H), 2.68 (s, 3H).

### Example 7—Synthesis of Compound 5: 1-(4-(6-(4-chlorophenyl)-2-phenylimidazo[1,2-a]pyridin-8-yl)phenyl)ethane-1-one

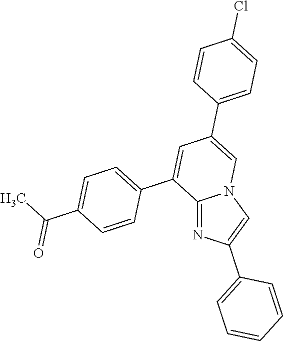 [SMILES: CC(=O)c1ccc(-c2cc(-c3ccc(Cl)cc3)cn3cc(-c4ccccc4)nc23)cc1]

A mixture of 2-amino-3-iodine-5-bromopyridine (1.00 mmol), 2-bromo-1-phenylethane-1-one (1.00 mmol) and sodium carbonate (1.00 mmol), in 20.0 mL of ethanol, was refluxed under stirring until the disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol) was then suspended in ethanol/water (4:1) and added with Pd(OAc)₂ (0.10 mmol), PPh₃ (0.20 mmol), 4-acetyl-phenylboronic acid (1.00 mmol) and Na₂CO₃ (2.00 mmol). The resulting mixture was refluxed under stirring until the disappearance of the starting material (TLC analysis). After cooling, it was evaporated to dryness under reduced pressure and the residue obtained is purified by column chromatography (silica gel, eluent mixture of ethyl acetate/petroleum ether). The 1-(4-(6-bromo-2-phenylimidazo[1,2-a]pyridin-8-yl)phenyl)ethane-1-one thus obtained (1.00 mmol) was then suspended in ethanol/water (4:1) and added with Pd(OAc)₂ (0.10 mmol), PPh₃ (0.20 mmol), 4-chlorophenylboronic acid (1.00 mmol) and sodium carbonate (2.00 mmol). The resulting mixture was refluxed under stirring until the disappearance of the starting material (TLC analysis). After cooling, it was evaporated to dryness under reduced pressure and the residue obtained was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 8.97 (d, J=1.7 Hz, 1H), 8.52 (s, 1H), 8.50 (s, 2H), 8.13 (d, J=8.3 Hz, 2H), 8.06-8.00 (m, 2H), 7.93-7.83 (m, 3H), 7.63-7.54 (m, 2H), 7.48 (t, J=7.5 Hz, 2H), 7.36 (t, J=7.4 Hz, 1H), 2.67 (s, 3H).

### Example 8—Synthesis of Compound 6: 1-(4-(6-(3,5-dimethoxyphenyl)-2-phenylimidazo[1,2-a]pyridin-8-yl)phenyl)ethane-1-one

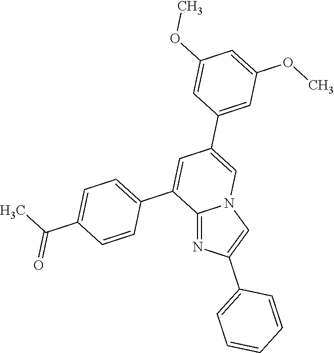 [SMILES: COc1cc(OC)cc(-c2cc(-c3ccc(C(C)=O)cc3)c3nc(-c4ccccc4)cn3c2)c1]

A mixture of 2-amino-3-iodine-5-bromopyridine (1.00 mmol), 2-bromo-1-phenylethane-1-one (1.00 mmol) and sodium carbonate (1.00 mmol), in 20.0 mL of ethanol, was refluxed under stirring until the disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol) was then suspended in ethanol/water (4:1) and added to Pd(OAc)₂ (0.10 mmol), PPh₃ (0.20 mmol), 4-acetyl-phenylboronic acid (1.00 mmol) and sodium carbonate 2.00 mmol). The resulting mixture was refluxed under stirring until the disappearance of the starting material (TLC analysis). After cooling, it was evaporated to dryness under reduced pressure and the residue obtained was purified by column chromatography (silica gel, eluent mixture of ethyl acetate/petroleum ether). The 1-(4-(6-bromo-2-phenylimidazo[1,2-a]pyridin-8-yl)phenyl)ethane-1-one thus obtained (1.00 mmol) was then suspended in ethanol/water (4:1) and added to Pd(OAc)₂ (0.10 mmol), PPh₃ (0.20 mmol), 3,5-dimethoxy-phenylboronic acid (1.00 mmol) and sodium carbonate (2.00 mmol). The resulting mixture was refluxed under stirring until the disappearance of the starting material (TLC analysis). After cooling, it was evaporated to dryness under reduced pressure and the residue obtained was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 8.98 (d, J=2.4 Hz, 1H), 8.55-8.46 (m, 3H), 8.18-8.09 (m, 2H), 8.07-8.00 (m, 2H), 7.89 (d, J=2.8 Hz, 1H), 7.52-7.43 (m, 2H), 7.36 (t, J=7.4 Hz, 1H), 7.01-6.94 (m, 2H), 6.57 (d, J=2.7 Hz, 1H), 3.85 (s, 6H), 2.67 (s, 3H).

### Example 9—Synthesis of Compound 7: methyl 4-(6-(4-cyanophenyl)-2-phenylimidazo[1,2-a]pyridin-8-yl)benzoate

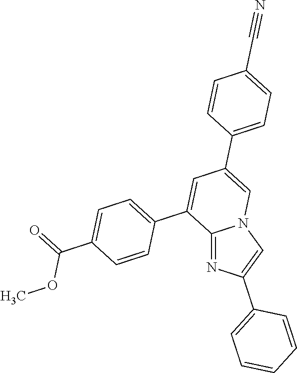 [SMILES: COC(=O)c1ccc(-c2cc(-c3ccc(C#N)cc3)cn3cc(-c4ccccc4)nc23)cc1]

A mixture of 2-amino-3-iodine-5-bromopyridine (1.00 mmol), 2-bromo-1-phenylethane-1-one (1.00 mmol) and sodium carbonate (1.00 mmol), in 20.0 mL of ethanol, was refluxed under stirring until the disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol) was then suspended in ethanol/water (4:1) and added to Pd(OAc)₂ (0.10 mmol), PPh₃ (0.20 mmol), (4-(methoxycarbonyl)phenyl)boronic acid (1.00 mmol) and sodium carbonate (2.00 mmol). The resulting mixture was refluxed under stirring until the disappearance of the starting material (TLC analysis). After cooling, it was evaporated to dryness under reduced pressure and the residue obtained was purified by column chromatography (silica gel, eluent mixture of ethyl acetate/petroleum ether). The methyl 4-(6-bromo-2-phenylimidazo[1,2-a]pyridin-8-yl)benzoate (1.00 mmol) thus obtained was then suspended in ethanol/water (4:1) and added to Pd(OAc)₂ (0.10 mmol), PPh₃ (0.20 mmol), 4-cyano-phenylboronic acid (1.00 mmol) and sodium carbonate (2.00 mmol). The resulting mixture was refluxed under stirring until the disappearance of the starting material (TLC analysis). After cooling, it was evaporated to dryness under reduced pressure and the residue obtained was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 9.13 (d, J=2.4 Hz, 1H), 8.54 (d, J=7.6 Hz, 2H), 8.15 (d, J=7.6 Hz, 2H), 8.11 (d, J=8.4 Hz, 2H), 8.06 (d, J=6.8 Hz, 2H), 8.02 (d, J=9.6 Hz, 2H), 7.99 (d, J=2.4 Hz, 2H), 7.49 (t, J=8.0 Hz, 2H), 7.37 (t, J=8.4 Hz, 1H), 3.31 (s, 3H).

### Example 10—Synthesis of Compound 8: 6,8-bis(4-chlorophenyl)-2-phenylimidazo[1,2-a]pyridine

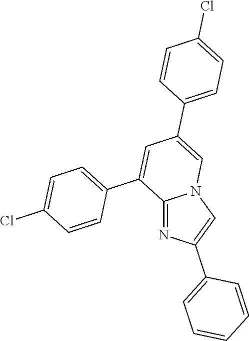 [SMILES: Clc1ccc(-c2cc(-c3ccc(Cl)cc3)c3nc(-c4ccccc4)cn3c2)cc1]

A mixture of 2-amino-3-iodine-5-bromopyridine (1.00 mmol), 2-bromo-1-phenylethane-1-one (1.00 mmol) and sodium carbonate (1.00 mmol) in 20.0 mL of ethanol was refluxed under stirring until the disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol), suspended in ethanol/water (4:1), was then added to Pd(OAc)₂ (0.10 mmol), PPh₃ (0.20 mmol), 4-chlorophenylboronic acid (2.00 mmol) and sodium carbonate (2.00 mmol) and the resulting mixture was refluxed under stirring until the starting material disappears (TLC analysis). After cooling, the reaction mixture was evaporated to dryness under reduced pressure and the residue was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 8.96 (d, J=1.9 Hz, 1H), 8.50 (d, J=1.8 Hz, 1H), 8.45-8.35 (m, 2H), 8.03 (d, J=7.6 Hz, 2H), 7.93-7.85 (m, 2H), 7.83 (d, J=1.9 Hz, 1H), 7.66-7.55 (m, 4H), 7.48 (t, J=7.6 Hz, 2H), 7.37 (d, J=7.3 Hz, 1H).

### Example 11—Synthesis of Compound 9: 2,6,8-triphenylimidazo[1,2-a]pyridine

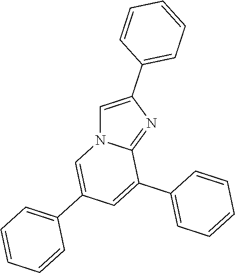 [SMILES: c1ccc(-c2cc(-c3ccccc3)c3nc(-c4ccccc4)cn3c2)cc1]

A mixture of 2-amino-3-iodine-5-bromopyridine (1.00 mmol), 2-bromo-1-phenylethane-1-one (1.00 mmol) and sodium carbonate (1.00 mmol) in 20.0 mL of ethanol was refluxed under stirring until the disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol), suspended in ethanol/water (4:1), was then added to Pd(OAc)₂ (0.10 mmol), PPh₃ (0.20 mmol), phenylboronic acid (2.00 mmol) and sodium carbonate (2.00 mmol) and the resulting mixture was refluxed under stirring until the starting material disappears (TLC analysis). After cooling, the reaction mixture was evaporated to dryness under reduced pressure and the residue was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 8.91 (d, 1H, J=1.6 Hz), 8.50 (s, 1H), 8.32 (d, 2H, J=7.2 Hz), 8.02 (d, 2H, 7.2 Hz), 7.85 (d, 2H, J=7.2 Hz), 7.75 (d, 2H, J=1.6 Hz), 7.58-7.41 (m, 8H), 7.36-7.33 (m, 1H).

### Example 12—Synthesis of Compound 10: 2-phenyl-6,8-di-p-tolylimidazo[1,2-a]pyridine

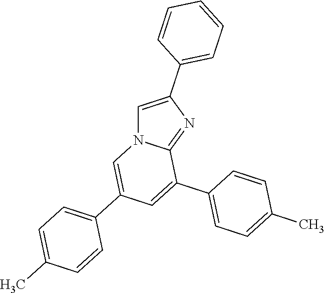

A mixture of 2-amino-3-iodine-5-bromopyridine (1.00 mmol), 2-bromo-1-phenylethane-1-one (1.00 mmol) and sodium carbonate (1.00 mmol) in 20.0 mL of ethanol was refluxed under stirring until disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol), suspended in ethanol/water (4:1), was then added to Pd(OAc)₂ (0.10 mmol), PPh₃ (0.20 mmol), p-tolylboronic acid (2.00 mmol) and sodium carbonate (2.00 mmol) and the resulting mixture was refluxed under stirring until the starting material disappears (TLC analysis). After cooling, the reaction mixture was evaporated to dryness under reduced pressure and the residue was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 8.84 (d, J=1.7 Hz, 1H), 8.47 (s, 1H), 8.22 (d, J=8.2 Hz, 2H), 8.04-7.96 (m, 2H), 7.75-7.68 (m, 3H), 7.47 (t, J=7.7 Hz, 2H), 7.40-7.29 (m, 5H), 2.41 (s, 3H), 2.38 (s, 3H).

### Example 13—Synthesis of Compound 11: 6,8-bis(4-methoxyphenyl)-2-phenylimidazo[1,2-a]pyridine

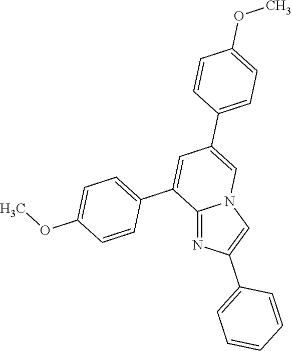 [SMILES: COc1ccc(-c2cc(-c3ccc(OC)cc3)c3nc(-c4ccccc4)cn3c2)cc1]

A mixture of 2-amino-3-iodine-5-bromopyridine (1.00 mmol), 2-bromo-1-phenylethane-1-one (1.00 mmol) and sodium carbonate (1.00 mmol) in 20.0 mL of ethanol was refluxed under stirring until disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol), suspended in ethanol/water (4:1), was then added to Pd(OAc)₂ (0.10 mmol), PPh₃ (0.20 mmol), (4-methoxyphenyl)boronic acid (2.00 mmol) and sodium carbonate (2.00 mmol) and the resulting mixture was refluxed under stirring until the starting material disappears (TLC analysis). After cooling, the reaction mixture was evaporated to dryness under reduced pressure and the residue was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 8.78 (d, J=1.9 Hz, 1H), 8.46 (d, J=1.3 Hz, 1H), 8.37-8.29 (m, 2H), 8.02 (d, J=7.7 Hz, 2H), 7.82-7.72 (m, 2H), 7.70 (d, J=1.8 Hz, 1H), 7.48 (t, J=7.4 Hz, 2H), 7.35 (t, J=7.3 Hz, 1H), 7.17-7.03 (m, 4H), 3.87 (s, 3H), 3.84 (s, 3H).

### Example 14—Synthesis of Compound 12: dimethyl 4,4′-(2-phenylimidazo[1,2-a]pyridine-6,8-diyl)dibenzaldehyde

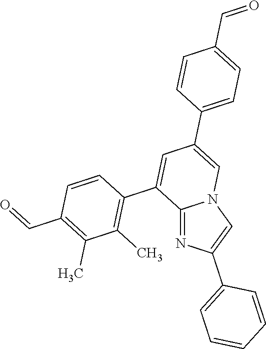 [SMILES: Cc1c(C=O)ccc(-c2cc(-c3ccc(C=O)cc3)cn3cc(-c4ccccc4)nc23)c1C]

A mixture of 2-amino-3-iodine-5-bromopyridine (1.00 mmol), 2-bromo-1-phenylethane-1-one (1.00 mmol) and sodium carbonate (1.00 mmol) in 20.0 mL of ethanol was refluxed under stirring until the disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol), suspended in ethanol/water (4:1), was then added to Pd(OAc)₂ (0.10 mmol), PPh₃ (0.20 mmol), (4-formylphenyl)boronic acid (2.00 mmol) and sodium carbonate (2.00 mmol) and the resulting mixture was refluxed under stirring until the starting material disappears (TLC analysis). After cooling, the reaction mixture was evaporated to dryness under reduced pressure and the residue was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 10.13 (s, 1H), 10.10 (s, 1H), 9.16 (s, 1H), 8.62 (d, 2H, J=8 Hz), 8.56 (s, 1H), 8.16-8.05 (m, 9H), 7.50 (t, 2H, J=7.6 Hz), 7.38 (m, 1H).

### Example 15—Synthesis of Compound 13: dimethyl 4,4′-(2-phenylimidazo[1,2-a]pyridine-6,8-diyl)dibenzoate

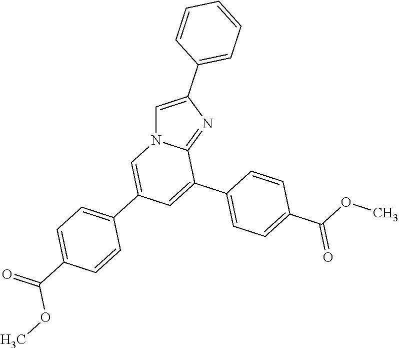 [SMILES: COC(=O)c1ccc(-c2cc(-c3ccc(C(=O)OC)cc3)c3nc(-c4ccccc4)cn3c2)cc1]

A mixture of 2-amino-3-iodine-5-bromopyridine (1.00 mmol), 2-bromo-1-phenylethane-1-one (1.00 mmol) and sodium carbonate (1.00 mmol) in 20.0 mL of ethanol was refluxed under stirring until the disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol), suspended in ethanol/water (4:1), was then added to Pd(OAc)₂ (0.10 mmol), PPh₃ (0.20 mmol), (4-(methoxycarbonyl)phenyl)boronic acid (2.00 mmol) and sodium carbonate (2.00 mmol) and the resulting mixture was refluxed under stirring until the starting material disappears (TLC analysis). After cooling, the reaction mixture was evaporated to dryness under reduced pressure and the residue was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 9.12 (d, J=1.8 Hz, 1H), 8.57-8.49 (m, 3H), 8.18-8.13 (m, 2H), 8.11 (d, J=8.5 Hz, 2H), 8.08-8.00 (m, 4H), 7.99 (d, J=1.8 Hz, 1H), 7.49 (t, J=7.7 Hz, 2H), 7.37 (t, J=7.3 Hz, 1H), 3.93 (s, 3H), 3.91 (s, 3H).

### Example 16—Synthesis of Compound 14: 1,1′-((2-(4-hydroxyphenyl)imidazo[1,2-a]pyridine-6,8-diyl)bis(4,1-phenylene))bis(ethan-1-one)

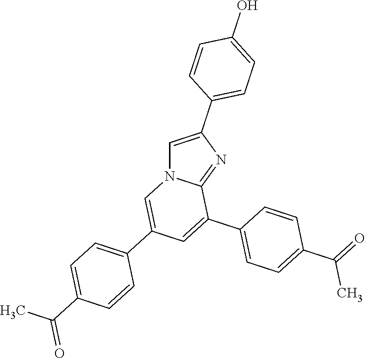 [SMILES: CC(=O)c1ccc(-c2cc(-c3ccc(C(C)=O)cc3)c3nc(-c4ccc(O)cc4)cn3c2)cc1]

A mixture of 2-amino-3-iodine-5-bromopyridine (1.00 mmol), 2-bromo-1-(4-hydroxyphenyl)ethan-1-one (1.00 mmol) and sodium carbonate (1.00 mmol) in 20.0 mL of ethanol was refluxed under stirring until the disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol), suspended in ethanol/water (4:1), was then added to Pd(OAc)₂ (0.10 mmol), PPh₃ (0.20 mmol), (4-acetylphenyl)boronic acid (2.00 mmol) and sodium carbonate (2.00 mmol) and the resulting mixture was refluxed under stirring until the starting material disappears (TLC analysis). After cooling, the reaction mixture was evaporated to dryness under reduced pressure and the residue was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 9.62 (s, 1H), 9.08 (s, 1H), 8.52 (d, J=8.1 Hz, 2H), 8.37 (s, 1H), 8.22-8.06 (m, 4H), 8.03 (d, J=8.2 Hz, 2H), 7.94 (s, 1H), 7.85 (d, J=8.2 Hz, 2H), 6.87 (d, J=8.2 Hz, 2H), 2.68 (s, 3H), 2.65 (s, 3H).

### Example 17—Synthesis of Compound 15: 1,1′-((2-(4-methoxyphenyl)imidazo[1,2-a]pyridine-6,8-diyl)bis(4,1-phenylene))bis(ethan-1-one)

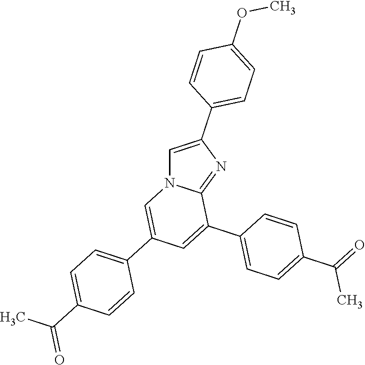 [SMILES: COc1ccc(-c2cn3cc(-c4ccc(C(C)=O)cc4)cc(-c4ccc(C(C)=O)cc4)c3n2)cc1]

A mixture of 2-amino-3-iodine-5-bromopyridine (1.00 mmol), 2-bromo-1-(4-methoxyphenyl)ethan-1-one (1.00 mmol) and sodium carbonate (1.00 mmol) in 20.0 mL of ethanol was refluxed under stirring until the disappearance of the starting material (TLC analysis). After cooling, the reaction mixture was evaporated to dryness and the resulting solid was purified by washing with water and subsequent crystallization from ethanol. The 2-phenyl-imidazo[1,2-a]pyridine thus obtained (1.00 mmol), suspended in ethanol/water (4:1), was then added to Pd(OAc)₂ (0.10 mmol), PPh₃ (0.20 mmol), (4-acetylphenyl)boronic acid (2.00 mmol) and sodium carbonate (2.00 mmol) and the resulting mixture was refluxed under stirring until the starting material disappears (TLC analysis). After cooling, the reaction mixture was evaporated to dryness under reduced pressure and the residue was purified by column chromatography (silica gel, eluent mixture of ethyl acetate/petroleum ether). The pure product was finally characterized by physicochemical and spectroscopic data. ¹H NMR (400 MHz, DMSO-d₆) δ (ppm): 9.08 (d, J=1.8 Hz, 1H), 8.54-8.47 (m, 2H), 8.44 (s, 1H), 8.18-8.12 (m, 2H), 8.12-8.07 (m, 2H), 8.03 (d, J=8.5 Hz, 2H), 8.00-7.90 (m, 3H), 7.09-6.97 (m, 2H), 3.81 (s, 3H), 2.67 (s, 3H), 2.65 (s, 3H).

### EQUIVALENTS

The invention is susceptible to variations falling within the scope of the inventive concept defined by the claims and as predictable based upon the disclosure given here. In this context, all the details can be replaced by equivalent elements and the materials, shapes, and dimensions as applicable can be any equivalent as predictable from what is given herein.

1. A compound of Formula I, or a pharmaceutically acceptable salt or prodrug thereof: 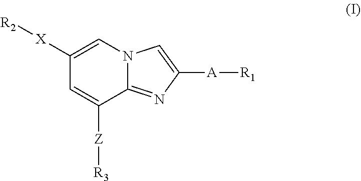
where A, X, and Z are each independently selected from aryl, heteroaryl, heterocyclic, or cyclic groups;
R₁ is selected from hydrogen, amino, fluoro, chloro, trifluoromethyl, cyano, nitro, carboxy, formyl, carbamoyl, acetyl, sulfamoyl, mercapto, acrylaldehyde, acrylate, acrylamide, acrylonitrile, dichloroacetamide, C1-C8 alkyl, C2-C8 alkenyl, C2-C8 alkynyl, C1-C6 alkoxy, C2-C6alkenyloxy, C2-C6alkynyloxy, C3-C7cycloalkyl, C3-C7heterocycloalkyl, hydroxyC1-C6alkyl, hydroxyC1-C6alkoxy, hydroxyC1-C6alkylamino, C1-C6alkoxyC1-C6alkyl, C1-C6alkoxyC1-C6alkylamino, aminoC1-C6alkyl, aminoC1-C6alkoxy, C1-C6alkylamino, di-C1-C6alkylamino, C1-C6-alkyldiamino, C1-C6alkoxycarbonyl, C1-C6alkoxyaminocarbonyl, C1-C6alkylthio, phosphateC1-C6alkylthio, sulfateC1-C6alkylthio, thioC1-C6alkyl, thioC1-C6alkoxy, thioC1-C6alkylamino, heterocycloC1-C6alkyl, heterocycloC1-C6alkoxy, heterocycloC1-C6alkylamino, phosphateC1-C6alkylamino, sulfateC1-C6alkylamino, C1-C6alkylsulfinyl, C1-C6alkylsulfonyl, C1-C6alkylthiocarboxamide, C1-C6alkylcarboxamide, C1-C6 alkylthioureyl, C1-C6alkylureyl, C1-C6dialkylamino, C1-C6alkoxycarbonyl, C1-C6dialkylcarboxamide, C1-C6 dialkylsulfonamide, C1-C6dialkylthiocarboxamido, C1-C6haloalkyl, C1-C6haloalkoxy, C1-C6guanidinealkyl, C1-C6cyanoalkyl, arylC1-C6alkoxy, heteroarylC1-C6alkoxy, cyclicC1-C6alkoxy, phosphateC1-C6alkoxy, sulfateC1-C6alkoxy, arylC1-C6alkylamino, heteroarylC1-C6alkylamino, cyclicC1-C6alkylamino, (((3R,4S,5S,6R)-3,4,5-trihydroxy-6-(hydroxymethyl)tetrahydro-2H-pyran-2-yl, aryl, heteroaryl, heterocyclic, and cyclic groups; and
R₂ and R₃ are each independently selected from hydrogen, halogen, fluoro, chloro, bromo, iodo, nitro, methyl, methoxy, cyano, formyl, carboxy, hydroxy, thiol, amino, amido, acetyl, sulfonamido, acryl, acrylaldehyde, but-3-en-2-one, methyl acrylate, N,N-dimethylacrylate, 2-(methylsulfonyl)vinyl, ethane-1-sulfonate, ethane-1-sulfonamido, acrylonitrile, nitrovinyl, pent-2-ene-1,4-dione, 4-oxobut-2-enoate, N,N-dimethyl-4-oxobut-2-enoate, ethan-1-one, and oxiran-2-yl-ethan-1-one, trifluoromethyl, carbamoyl, sulfamoyl, acrylamide, dichloroacetamide, C1-C8 alkyl, C2-C8 alkenyl, C2-C8 alkynyl, C1-C6 alkoxy, C2-C6alkenyloxy, C2-C6alkynyloxy, C3-C7cycloalkyl, C3-C7heterocycloalkyl, hydroxyC1-C6alkyl, hydroxyC1-C6alkoxy, hydroxyC1-C6alkylamino, C1-C6alkoxyC1-C6alkyl, C1-C6alkoxyC1-C6alkylamino, aminoC1-C6alkyl, aminoC1-C6alkoxy, C1-C6alkylamino, di-C1-C6alkylamino, C1-C6-alkyldiamino, C1-C6alkoxycarbonyl, C1-C6alkoxyaminocarbonyl, C1-C6alkylthio, phosphateC1-C6alkylthio, sulfateC1-C6alkylthio, thioC1-C6alkyl, thioC1-C6alkoxy, thioC1-C6alkylamino, heterocycloC1-C6alkyl, heterocycloC1-C6alkoxy, heterocycloC1-C6alkylamino, phosphateC1-C6alkylamino, sulfateC1-C6alkylamino, C1-C6alkylsulfinyl, C1-C6alkylsulfonyl, C1-C6alkylthiocarboxamide, C1-C6alkylcarboxamide, C1-C6 alkylthioureyl, C1-C6alkylureyl, C1-C6dialkylamino, C1-C6alkoxycarbonyl, C1-C6dialkylcarboxamide, C1-C6 dialkylsulfonamide, C1-C6dialkylthiocarboxamido, C1-C6haloalkyl, C1-C6haloalkoxy, C1-C6guanidinealkyl, C1-C6cyanoalkyl, arylC1-C6alkoxy, heteroarylC1-C6alkoxy, cyclicC1-C6alkoxy, phosphateC1-C6alkoxy, sulfateC1-C6alkoxy, arylC1-C6alkylamino, heteroarylC1-C6alkylamino, cyclicC1-C6alkylamino, (((3R,4S,5S,6R)-3,4,5-trihydroxy-6-(hydroxymethyl)tetrahydro-2H-pyran-2-yl, aryl, heteroaryl, heterocyclic, and cyclic groups;
and in which extant aryl, heteroaryl, heterocyclic, and cyclic groups are further functionalized with zero or more substituents selected from hydrogen, halogen, fluoro, chloro, bromo, iodo, nitro, methyl, methoxy, cyano, formyl, carboxy, hydroxy, thiol, amino, amido, acetyl, sulfonamido, acryl, acrylaldehyde, but-3-en-2-one, methyl acrylate, N,N-dimethylacrylate, 2-(methylsulfonyl)vinyl, ethane-1-sulfonate, ethane-1-sulfonamido, acrylonitrile, nitrovinyl, pent-2-ene-1,4-dione, 4-oxobut-2-enoate, N,N-dimethyl-4-oxobut-2-enoate, ethan-1-one, and oxiran-2-yl-ethan-1-one, trifluoromethyl, carbamoyl, sulfamoyl, acrylamide, acrylonitrile, dichloroacetamide, C1-C6 alkyl, C2-C8 alkenyl, C2-C8 alkynyl, C1-C6 alkoxy, C2-C6 alkenyloxy, C2-C6 alkynyloxy, C1-C6 alkylcarboamino, C1-C6 alkylthio, C1-C6 alkylsulfonyl, C1-C6 alkylsulfonyl, C1-C6 alkylamino, di-C1-C6 alkylamino, amino C1-C6 alkyl, C3-C7 cycloalkyl, C1-C6 alkylcarbonyl, C2-C6 alkenylcarbonyl, C2-C6 alkynylcarbonyl, C1-C6 alkylaminocarbonyl, C1-C6 dialkyl aminocarbonyl, C1-C6 alkylsulfonyl aminocarbonyl, C1-C6 alkoxycarbonyl, arylaminocarbonyl and C3-C7 cycloalkylaminocarbonyl.

2. The compound of claim 1 in which A, X, and Z are each phenyl; R₁ is selected from hydrogen, fluoro, chloro, bromo, iodo, nitro, methyl, methoxy, cyano, formyl, carboxy, thiol, amino, amido, acetyl, sulfonamido, acryl, acrylaldehyde, but-3-en-2-one, methyl acrylate, N,N-dimethylacrylate, 2-(methylsulfonyl)vinyl, ethane-1-sulfonate, ethane-1-sulfonamido, acrylonitrile, nitrovinyl, pent-2-ene-1,4-dione, 4-oxobut-2-enoate, N,N-dimethyl-4-oxobut-2-enoate, ethan-1-one, and oxiran-2-yl-ethan-1-one; and R₂ and R₃ are each independently selected from hydrogen, halogen, fluoro, chloro, bromo, iodo, nitro, methyl, methoxy, cyano, formyl, carboxy, hydroxy, thiol, amino, amido, acetyl, sulfonamido, acryl, acrylaldehyde, but-3-en-2-one, methyl acrylate, N,N-dimethylacrylate, 2-(methylsulfonyl)vinyl, ethane-1-sulfonate, ethane-1-sulfonamido, acrylonitrile, nitrovinyl, pent-2-ene-1,4-dione, 4-oxobut-2-enoate, N,N-dimethyl-4-oxobut-2-enoate, ethan-1-one, and oxiran-2-yl-ethan-1-one.

3. The compound of claim 1 in which A, X, and Z are each phenyl; R₁ is selected from hydrogen, acetyl, formyl, alkyl, methyl, methyl ester (in either direction), methyl ether, fluoro, chloro, alkoxy, methoxy, and cyano; and R₂ and R₃ are each independently selected from hydrogen, acetyl, formyl, alkyl, methyl, methyl ester (in either direction), methyl ether, halogen, fluoro, chloro, hydroxy, alkoxy, methoxy, and cyano.

4. The compound of claim 3 in which extant aryl, heteroaryl, heterocyclic, and cyclic groups are further functionalized with up to two additional substituents selected from hydrogen, acetyl, formyl, alkyl, methyl, methyl ester (in either direction), methyl ether, halogen, fluoro, chloro, hydroxy, alkoxy, methoxy, and cyano.

5. The compound of claim 1 where the compound is selected from 2,6,8-triphenylimidazo[1,2-a]pyridine, 2-(4-chlorophenyl)-6,8-diphenylimidazo[1,2-a]pyridine, 2-(3-chlorophenyl)-6,8-diphenylimidazo[1,2-a]pyridine, 2-(2-chlorophenyl)-6,8-diphenylimidazo[1,2-a]pyridine, 2-(3-bromophenyl)-6,8-diphenylimidazo[1,2-a]pyridine, 2-(2-bromophenyl)-6,8-diphenylimidazo[1,2-a]pyridine, 2-(4-iodophenyl)-6,8-diphenylimidazo[1,2-a]pyridine, 2-(3-iodophenyl)-6,8-diphenylimidazo[1,2-a]pyridine, 2-(2-iodophenyl)-6,8-diphenylimidazo[1,2-a]pyridine, 2-(4-nitrophenyl)-6,8-diphenylimidazo[1,2-a]pyridine, 2-(3-nitrophenyl)-6,8-diphenylimidazo[1,2-a]pyridine, 2-(2-nitrophenyl)-6,8-diphenylimidazo[1,2-a]pyridine, 6,8-diphenyl-2-(p-tolyl)imidazo[1,2-a]pyridine, 6,8-diphenyl-2-(m-tolyl)imidazo[1,2-a]pyridine, 6,8-diphenyl-2-(o-tolyl)imidazo[1,2-a]pyridine, 2-(4-methoxyphenyl)-6,8-diphenylimidazo[1,2-a]pyridine, 2-(3-methoxyphenyl)-6,8-diphenylimidazo[1,2-a]pyridine, 2-(2-methoxyphenyl)-6,8-diphenylimidazo[1,2-a]pyridine, 4-(6,8-diphenylimidazo[1,2-a]pyridin-2-yl)benzonitrile, 3-(6,8-diphenylimidazo[1,2-a]pyridin-2-yl)benzonitrile, 2-(6,8-diphenylimidazo[1,2-a]pyridin-2-yl)benzonitrile, 4-(6,8-diphenylimidazo[1,2-a]pyridin-2-yl)benzaldehyde, 3-(6,8-diphenylimidazo[1,2-a]pyridin-2-yl)benzaldehyde, 2-(6,8-diphenylimidazo[1,2-a]pyridin-2-yl)benzaldehyde, 4-(6,8-diphenylimidazo[1,2-a]pyridin-2-yl)phenol, 3-(6,8-diphenylimidazo[1,2-a]pyridin-2-yl)phenol, 2-(6,8-diphenylimidazo[1,2-a]pyridin-2-yl)phenol, 4-(6,8-diphenylimidazo[1,2-a]pyridin-2-yl)benzenethiol, 3-(6,8-diphenylimidazo[1,2-a]pyridin-2-yl)benzenethiol, 2-(6,8-diphenylimidazo[1,2-a]pyridin-2-yl)benzenethiol, 4-(6,8-diphenylimidazo[1,2-a]pyridin-2-yl)aniline, 3-(6,8-diphenylimidazo[1,2-a]pyridin-2-yl)aniline, 2-(6,8-diphenylimidazo[1,2-a]pyridin-2-yl)aniline, 4-(6,8-diphenylimidazo[1,2-a]pyridin-2-yl)benzamide, 3-(6,8-diphenylimidazo[1,2-a]pyridin-2-yl)benzamide, 2-(6,8-diphenylimidazo[1,2-a]pyridin-2-yl)benzamide, 4-(6,8-diphenylimidazo[1,2-a]pyridin-2-yl)benzenesulfonamide, 3-(6,8-diphenylimidazo[1,2-a]pyridin-2-yl)benzenesulfonamide, 2-(6,8-diphenylimidazo[1,2-a]pyridin-2-yl)benzenesulfonamide, 3-(4-(6,8-diphenylimidazo[1,2-a]pyridin-2-yl)phenyl)acrylaldehyde, 4-(4-(6,8-diphenylimidazo[1,2-a]pyridin-2-yl)phenyl)but-3-en-2-one, methyl 3-(4-(6,8-diphenylimidazo[1,2-a]pyridin-2-yl)phenyl)acrylate, 3-(4-(6,8-diphenylimidazo[1,2-a]pyridin-2-yl)phenyl)-N,N-dimethylacrylamide, 2-(4-(2-(methylsulfonyl)vinyl)phenyl)-6,8-diphenylimidazo[1,2-a]pyridinemethyl, 2-(4-(6,8-diphenylimidazo[1,2-a]pyridin-2-yl)phenyl)ethene-1-sulfonate, 2-(4-(6,8-diphenylimidazo[1,2-a]pyridin-2-yl)phenyl)ethene-1-sulfonamide, 3-(4-(6,8-diphenylimidazo[1,2-a]pyridin-2-yl)phenyl)acrylonitrile, 2-(4-(2-nitrovinyl)phenyl)-6,8-diphenylimidazo[1,2-a]pyridine, 1-(4-(6,8-diphenylimidazo[1,2-a]pyridin-2-yl)phenyl)pent-2-ene-1,4-dione, methyl 4-(4-(6,8-diphenylimidazo[1,2-a]pyridin-2-yl)phenyl)-4-oxobut-2-enoate, 4-(4-(6,8-diphenylimidazo[1,2-a]pyridin-2-yl)phenyl)-N,N-dimethyl-4-oxobut-2-enamide, 1-(3-(4-(6,8-diphenylimidazo[1,2-a]pyridin-2-yl)benzoyl)oxirane-2-yl)ethan-1-one, 6-(4-chlorophenyl)-2,8-diphenylimidazo[1,2-a]pyridine, 6-(3-chlorophenyl)-2,8-diphenylimidazo[1,2-a]pyridine, 6-(2-chlorophenyl)-2,8-diphenylimidazo[1,2-a]pyridine, 6-(4-bromophenyl)-2,8-diphenylimidazo[1,2-a]pyridine, 6-(3-bromophenyl)-2,8-diphenylimidazo[1,2-a]pyridine, 6-(2-bromophenyl)-2,8-diphenylimidazo[1,2-a]pyridine, 6-(4-iodophenyl)-2,8-diphenylimidazo[1,2-a]pyridine, 6-(3-iodophenyl)-2,8-diphenylimidazo[1,2-a]pyridine, 6-(2-iodophenyl)-2,8-diphenylimidazo[1,2-a]pyridine, 6-(4-nitrophenyl)-2,8-diphenylimidazo[1,2-a]pyridine, 6-(3-nitrophenyl)-2,8-diphenylimidazo[1,2-a]pyridine, 6-(2-nitrophenyl)-2,8-diphenylimidazo[1,2-a]pyridine, 2,8-diphenyl-6-(p-tolyl)imidazo[1,2-a]pyridine, 2,8-diphenyl-6-(m-tolyl)imidazo[1,2-a]pyridine, 2,8-diphenyl-6-(o-tolyl)imidazo[1,2-a]pyridine, 6-(4-methoxyphenyl)-2,8-diphenylimidazo[1,2-a]pyridine, 6-(3-methoxyphenyl)-2,8-diphenylimidazo[1,2-a]pyridine, 6-(2-methoxyphenyl)-2,8-diphenylimidazo[1,2-a]pyridine, 4-(2,8-diphenylimidazo[1,2-a]pyridin-6-yl)benzonitrile, 3-(2,8-diphenylimidazo[1,2-a]pyridin-6-yl)benzonitrile, 2-(2,8-diphenylimidazo[1,2-a]pyridin-6-yl)benzonitrile, 4-(2,8-diphenylimidazo[1,2-a]pyridin-6-yl)benzaldehyde, 3-(2,8-diphenylimidazo[1,2-a]pyridin-6-yl)benzaldehyde, 2-(2,8-diphenylimidazo[1,2-a]pyridin-6-yl)benzaldehyde, 4-(2,8-diphenylimidazo[1,2-a]pyridin-6-yl)phenol, 3-(2,8-diphenylimidazo[1,2-a]pyridin-6-yl)phenol, 2-(2,8-diphenylimidazo[1,2-a]pyridin-6-yl)phenol, 4-(2,8-diphenylimidazo[1,2-a]pyridin-6-yl)benzenethiol, 3-(2,8-diphenylimidazo[1,2-a]pyridin-6-yl)benzenethiol, 2-(2,8-diphenylimidazo[1,2-a]pyridin-6-yl)benzenethiol, 4-(2,8-diphenylimidazo[1,2-a]pyridin-6-yl)aniline, 3-(2,8-diphenylimidazo[1,2-a]pyridin-6-yl)aniline, 2-(2,8-diphenylimidazo[1,2-a]pyridin-6-yl)aniline, 4-(2,8-diphenylimidazo[1,2-a]pyridin-6-yl)benzamide, 3-(2,8-diphenylimidazo[1,2-a]pyridin-6-yl)benzamide, 2-(2,8-diphenylimidazo[1,2-a]pyridin-6-yl)benzamide, 4-(2,8-diphenylimidazo[1,2-a]pyridin-6-yl)benzenesulfonamide, 3-(2,8-diphenylimidazo[1,2-a]pyridin-6-yl)benzenesulfonamide, 2-(2,8-diphenylimidazo[1,2-a]pyridin-6-yl)benzenesulfonamide, 3-(4-(2,8-diphenylimidazo[1,2-a]pyridin-6-yl)phenyl)acrylaldehyde, 4-(4-(2,8-diphenylimidazo[1,2-a]pyridin-6-yl)phenyl)but-3-en-2-one, methyl 3-(4-(2,8-diphenylimidazo[1,2-a]pyridin-6-yl)phenyl)acrylate, 3-(4-(2,8-diphenylimidazo[1,2-a]pyridin-6-yl)phenyl)-N,N-dimethylacrylamide, 6-(4-(2-(methylsulfonyl)vinyl)phenyl)-2,8-diphenylimidazo[1,2-a]pyridine, methyl 2-(4-(2,8-diphenylimidazo[1,2-a]pyridin-6-yl)phenyl)ethene-1-sulfonate, 2-(4-(2,8-diphenylimidazo[1,2-a]pyridin-6-yl)phenyl)ethene-1-sulfonamide, 3-(4-(2,8-diphenylimidazo[1,2-a]pyridin-6-yl)phenyl)acrylonitrile, 6-(4-(2-nitrovinyl)phenyl)-2,8-diphenylimidazo[1,2-a]pyridine, 1-(4-(2,8-diphenylimidazo[1,2-a]pyridin-6-yl)phenyl)pent-2-ene-1,4-dione, methyl 4-(4-(2,8-diphenylimidazo[1,2-a]pyridin-6-yl)phenyl)-4-oxobut-2-enoate, 4-(4-(2,8-diphenylimidazo[1,2-a]pyridin-6-yl)phenyl)-N,N-dimethyl-4-oxobut-2-enamide, 1-(3-(4-(2,8-diphenylimidazo[1,2-a]pyridin-6-yl)benzoyl)oxirane-2-yl)ethan-1-one, 8-(4-chlorophenyl)-2,6-diphenylimidazo[1,2-a]pyridine, 8-(3-chlorophenyl)-2,6-diphenylimidazo[1,2-a]pyridine, 8-(2-chlorophenyl)-2,6-diphenylimidazo[1,2-a]pyridine, 8-(4-bromophenyl)-2,6-diphenylimidazo[1,2-a]pyridine, 8-(3-bromophenyl)-2,6-diphenylimidazo[1,2-a]pyridine, 8-(2-bromophenyl)-2,6-diphenylimidazo[1,2-a]pyridine, 8-(4-iodophenyl)-2,6-diphenylimidazo[1,2-a]pyridine, 8-(3-iodophenyl)-2,6-diphenylimidazo[1,2-a]pyridine, 8-(2-iodophenyl)-2,6-diphenylimidazo[1,2-a]pyridine, 8-(4-nitrophenyl)-2,6-diphenylimidazo[1,2-a]pyridine, 8-(3-nitrophenyl)-2,6-diphenylimidazo[1,2-a]pyridine, 8-(2-nitrophenyl)-2,6-diphenylimidazo[1,2-a]pyridine, 2,6-diphenyl-8-(p-tolyl)imidazo[1,2-a]pyridine, 2,6-diphenyl-8-(m-tolyl)imidazo[1,2-a]pyridine, 2,6-diphenyl-8-(o-tolyl)imidazo[1,2-a]pyridine, 8-(4-methoxyphenyl)-2,6-diphenylimidazo[1,2-a]pyridine, 8-(3-methoxyphenyl)-2,6-diphenylimidazo[1,2-a]pyridine, 8-(2-methoxyphenyl)-2,6-diphenylimidazo[1,2-a]pyridine, 4-(2,6-diphenylimidazo[1,2-a]pyridin-8-yl)benzonitrile, 3-(2,6-diphenylimidazo[1,2-a]pyridin-8-yl)benzonitrile, 2-(2,6-diphenylimidazo[1,2-a]pyridin-8-yl)benzonitrile, 4-(2,6-diphenylimidazo[1,2-a]pyridin-8-yl)benzaldehyde, 3-(2,6-diphenylimidazo[1,2-a]pyridin-8-yl)benzaldehyde, 2-(2,6-diphenylimidazo[1,2-a]pyridin-8-yl)benzaldehyde, 4-(2,6-diphenylimidazo[1,2-a]pyridin-8-yl)phenol, 3-(2,6-diphenylimidazo[1,2-a]pyridin-8-yl)phenol, 2-(2,6-diphenylimidazo[1,2-a]pyridin-8-yl)phenol, 4-(2,6-diphenylimidazo[1,2-a]pyridin-8-yl)benzenethiol, 3-(2,6-diphenylimidazo[1,2-a]pyridin-8-yl)benzenethiol, 2-(2,6-diphenylimidazo[1,2-a]pyridin-8-yl)benzenethiol, 4-(2,6-diphenylimidazo[1,2-a]pyridin-8-yl)aniline, 3-(2,6-diphenylimidazo[1,2-a]pyridin-8-yl)aniline, 2-(2,6-diphenylimidazo[1,2-a]pyridin-8-yl)aniline, 4-(2,6-diphenylimidazo[1,2-a]pyridin-8-yl)benzamide, 3-(2,6-diphenylimidazo[1,2-a]pyridin-8-yl)benzamide, 2-(2,6-diphenylimidazo[1,2-a]pyridin-8-yl)benzamide, 4-(2,6-diphenylimidazo[1,2-a]pyridin-8-yl)benzenesulfonamide, 3-(2,6-diphenylimidazo[1,2-a]pyridin-8-yl)benzenesulfonamide, 2-(2,6-diphenylimidazo[1,2-a]pyridin-8-yl)benzenesulfonamide, 3-(4-(2,6-diphenylimidazo[1,2-a]pyridin-8-yl)phenyl)acrylaldehyde, 4-(4-(2,6-diphenylimidazo[1,2-a]pyridin-8-yl)phenyl)but-3-en-2-one, methyl 3-(4-(2,6-diphenylimidazo[1,2-a]pyridin-8-yl)phenyl)acrylate, 3-(4-(2,6-diphenylimidazo[1,2-a]pyridin-8-yl)phenyl)-N,N-dimethylacrylamide, 8-(4-(2-(methylsulfonyl)vinyl)phenyl)-2,6-diphenylimidazo[1,2-a]pyridine, methyl 2-(4-(2,6-diphenylimidazo[1,2-a]pyridin-8-yl)phenyl)ethene-1-sulfonate, 2-(4-(2,6-diphenylimidazo[1,2-a]pyridin-8-yl)phenyl)ethene-1-sulfonamide, 3-(4-(2,6-diphenylimidazo[1,2-a]pyridin-8-yl)phenyl)acrylonitrile, 8-(4-(2-nitrovinyl)phenyl)-2,6-diphenylimidazo[1,2-a]pyridine, 1-(4-(2,6-diphenylimidazo[1,2-a]pyridin-8-yl)phenyl)pent-2-ene-1,4-dione, methyl 4-(4-(2,6-diphenylimidazo[1,2-a]pyridin-8-yl)phenyl)-4-oxobut-2-enoate, 4-(4-(2,6-diphenylimidazo[1,2-a]pyridin-8-yl)phenyl)-N,N-dimethyl-4-oxobut-2-enamide, 1-(3-(4-(2,6-diphenylimidazo[1,2-a]pyridin-8-yl)benzoyl)oxirane-2-yl)ethan-1-one, 1,1′-((2-phenylimidazo[1,2-a]pyridine-6,8-diyl)bis(4,1-phenylene))bis(ethan-1-one), 4-(8-(4-acetylphenyl)-2-phenylimidazo[1,2-a]pyridin-6-yl)benzonitrile, 4-(8-(4-acetylphenyl)-2-phenylimidazo[1,2-a]pyridin-6-yl)benzonitrile, 1-(4-(6-(4-fluorophenyl)-2-phenylimidazo[1,2-a]pyridin-8-yl)phenyl)ethan-1-one, 1-(4-(6-(4-chlorophenyl)-2-phenylimidazo[1,2-a]pyridin-8-yl)phenyl)ethan-1-one, 1-(4-(6-(4-bromophenyl)-2-phenylimidazo[1,2-a]pyridin-8-yl)phenyl)ethan-1-one, 1-(4-(6-(3,5-dimethoxyphenyl)-2-phenylimidazo[1,2-a]pyridin-8-yl)phenyl)ethan-1-one, dimethyl 4,4′-(2-phenylimidazo[1,2-a]pyridine-6,8-diyl)dibenzoate, methyl 4-(6-(4-cyanophenyl)-2-phenylimidazo[1,2-a]pyridin-8-yl)benzoate, methyl 4-(6-(3-cyanophenyl)-2-phenylimidazo[1,2-a]pyridin-8-yl)benzoate, methyl 4-(6-(4-fluorophenyl)-2-phenylimidazo[1,2-a]pyridin-8-yl)benzoate, methyl 4-(6-(4-chlorophenyl)-2-phenylimidazo[1,2-a]pyridin-8-yl)benzoate, methyl 4-(6-(4-bromophenyl)-2-phenylimidazo[1,2-a]pyridin-8-yl)benzoate, methyl 4-(6-(3,5-dimethoxyphenyl)-2-phenylimidazo[1,2-a]pyridin-8-yl)benzoate, 6,8-bis(4-chlorophenyl)-2-phenylimidazo[1,2-a]pyridine, 4-(8-(4-chlorophenyl)-2-phenylimidazo[1,2-a]pyridin-6-yl)benzonitrile, 3-(8-(4-chlorophenyl)-2-phenylimidazo[1,2-a]pyridin-6-yl)benzonitrile, 8-(4-chlorophenyl)-6-(4-fluorophenyl)-2-phenylimidazo[1,2-a]pyridine, 6-(4-bromophenyl)-8-(4-chlorophenyl)-2-phenylimidazo[1,2-a]pyridine, 8-(4-chlorophenyl)-6-(3,5-dimethoxyphenyl)-2-phenylimidazo[1,2-a]pyridine, 1-(4-(8-(4-chlorophenyl)-2-phenylimidazo[1,2-a]pyridin-6-yl)phenyl)ethan-1-one, 3-(8-(4-acetylphenyl)-2-(4-(2-hydroxyethoxy)phenyl)imidazo[1,2-a]pyridin-6-yl)benzonitrile, 1-(4-(2-(4-((2-hydroxyethyl)amino)phenyl)-6-phenylimidazo[1,2-a]pyridin-8-yl)phenyl)ethan-1-one, 3-(8-(4-acetylphenyl)-2-(4-((2-hydroxyethyl)amino)phenyl)imidazo[1,2-a]pyridin-6-yl)benzonitrile, 1-(4-(6-(3,5-dimethoxyphenyl)-2-(4-((2-hydroxyethyl)amino)phenyl)imidazo[1,2-a]pyridin-8-yl)phenyl)ethan-1-one, 3-(8-(4-acetylphenyl)-2-(4-((2-morpholinoethyl)amino)phenyl)imidazo[1,2-a]pyridin-6-yl)benzonitrile, 3-(8-(4-acetylphenyl)-2-(4-((14-hydroxy-3,6,9,12-tetraoxatetradecyl)amino)phenyl)imidazo[1,2-a]pyridin-6-yl)benzonitrile3-(8-(4-acetylphenyl)-2-(4-((14-hydroxy-3,6,9,12-tetraoxatetradecyl)oxy)phenyl)imidazo[1,2-a]pyridin-6-yl)benzonitrile, 1-(4-(6-(3,5-dimethoxyphenyl)-2-(4-((2-morpholinoethyl)amino)phenyl)imidazo[1,2-a]pyridin-8-yl)phenyl)ethan-1-one, 1-(4-(6-(3,5-dimethoxyphenyl)-2-(4-((14-hydroxy-3,6,9,12-tetraoxatetradecyl)amino)phenyl)imidazo[1,2-a]pyridin-8-yl)phenyl)ethan-1-one, 1-(4-(6-(3,5-dimethoxyphenyl)-2-(4-((14-hydroxy-3,6,9,12-tetraoxatetradecyl)oxy)phenyl)imidazo[1,2-a]pyridin-8-yl)phenyl)ethan-1-one, 3-(8-(4-acetylphenyl)-2-(4-(2-morpholinoethoxy)phenyl)imidazo[1,2-a]pyridin-6-yl)benzonitrile1-(4-(6-(3,5-dimethoxyphenyl)-2-(4-(2-morpholinoethoxy)phenyl)imidazo[1,2-a]pyridin-8-yl)phenyl)ethan-1-one, 1-(4-(2-(4-(2-(diethylamino)ethoxy)phenyl)-6-(3,5-dimethoxyphenyl)imidazo[1,2-a]pyridin-8-yl)phenyl)ethan-1-one, 3-(8-(4-acetylphenyl)-2-(4-(2-(diethylamino)ethoxy)phenyl)imidazo[1,2-a]pyridin-6-yl)benzonitrile, 1-(4-(6-(3,5-dimethoxyphenyl)-2-(4-((2-(piperazin-1-yl)ethyl)amino)phenyl)imidazo[1,2-a]pyridin-8-yl)phenyl)ethan-1-one, 3-(8-(4-acetylphenyl)-2-(4-((2-(piperazin-1-yl)ethyl)amino)phenyl)imidazo[1,2-a]pyridin-6-yl)benzonitrile, 3-(8-(4-acetylphenyl)-2-(4-(2-(piperazin-1-yl)ethoxy)phenyl)imidazo[1,2-a]pyridin-6-yl)benzonitrile, 3-(8-(4-acetylphenyl)-2-(4-((2-(4-methylpiperazin-1-yl)ethyl)amino)phenyl)imidazo[1,2-a]pyridin-6-yl)benzonitrile, 1-(4-(6-(3,5-dimethoxyphenyl)-2-(4-(2-(piperazin-1-yl)ethoxy)phenyl)imidazo[1,2-a]pyridin-8-yl)phenyl)ethan-1-one, 1-(4-(6-(3,5-dimethoxyphenyl)-2-(4-((2-(4-methylpiperazin-1-yl)ethyl)amino)phenyl)imidazo[1,2-a]pyridin-8-yl)phenyl)ethan-1-one, N-(4-(8-(4-acetylphenyl)-6-(3,5-dimethoxyphenyl)imidazo[1,2-a]pyridin-2-yl)phenyl)-2-aminoacetamide, N-(4-(8-(4-acetylphenyl)-6-(3-cyanophenyl)imidazo[1,2-a]pyridin-2-yl)phenyl)-2-aminoacetamide, 4-(8-(4-acetylphenyl)-6-(3-cyanophenyl)imidazo[1,2-a]pyridin-2-yl)phenyl acetate, 4-(8-(4-acetylphenyl)-6-(3,5-dimethoxyphenyl)imidazo[1,2-a]pyridin-2-yl)phenyl acetate, 1-(4-(6-(3,5-dimethoxyphenyl)-2-(4-(2-(((3R,4S,5S,6R)-3,4,5-trihydroxy-6-(hydroxymethyl)tetrahydro-2H-pyran-2-yl)amino)ethoxy)phenyl)imidazo[1,2-a]pyridin-8-yl)phenyl)ethan-1-one, 3-(8-(4-acetylphenyl)-2-(4-(2-(((3R,4S,5S,6R)-3,4,5-trihydroxy-6-(hydroxymethyl)tetrahydro-2H-pyran-2-yl)amino)ethoxy)phenyl)imidazo[1,2-a]pyridin-6-yl)benzonitrile, 1,1′-((2-phenylimidazo[1,2-a]pyridine-6,8-diyl)bis(4,1-phenylene))bis(ethan-1-one), 3-(8-(4-acetylphenyl)-2-phenylimidazo[1,2-a]pyridin-6-yl)benzonitrile, 1-(4-(6-(4-fluorophenyl)-2-phenylimidazo[1,2-a]pyridin-8-yl)phenyl)ethane-1-one, 1-(4-(6-(4-chlorophenyl)-2-phenylimidazo[1,2-a]pyridin-8-yl)phenyl)ethane-1-one, 1-(4-(6-(3,5-dimethoxyphenyl)-2-phenylimidazo[1,2-a]pyridin-8-yl)phenyl)ethane-1-one, 6,8-bis(4-chlorophenyl)-2-phenylimidazo[1,2-a]pyridine, 2-phenyl-6,8-di-p-tolylimidazo[1,2-a]pyridine, 6,8-bis(4-methoxyphenyl)-2-phenylimidazo[1,2-a]pyridine, 4,4′-(2-phenylimidazo[1,2-a]pyridine-6,8-diyl)dibenzaldehyde, dimethyl 4,4′-(2-phenylimidazo[1,2-a]pyridine-6,8-diyl)dibenzoate, 1,1′-((2-(4-hydroxyphenyl)imidazo[1,2-a]pyridine-6,8-diyl)bis(4,1-phenylene))bis(ethan-1-one), and 1,1′-((2-(4-methoxyphenyl)imidazo[1,2-a]pyridine-6,8-diyl)bis(4,1-phenylene))bis(ethan-1-one).

6. The compound of claim 1 in which the compound is 1,1′-((2-phenylimidazo[1,2-a]pyridine-6,8-diyl)bis(4,1-phenylene))bis(ethan-1-one).

7. The compound of claim 1 in which the compound is 4-(8-(4-acetylphenyl)-2-phenylimidazo[1,2-a]pyridin-6-yl)benzonitrile.

8. A method of manufacture of a 2,6,8-trisubstituted imidazo[1,2-a]pyridine, the method comprising reacting an alpha-aminopyridine directly connected to bromine and to iodine with a beta-carbonyl alkyl halide directly connected to a first functional group to produce an imidazo[1,2-a]pyridine nucleus directly connected to the first functional group, then reacting the product sequentially with a boronic acid directly connected to a second functional group and a boronic acid directly connected to a third functional group, wherein the 2,6,8-trisubstituted imidazo[1,2-a]pyridine results.

9. The method of manufacture of claim 8 in which the resulting compound is 1,1′-((2-phenylimidazo[1,2-a]pyridine-6,8-diyl)bis(4,1-phenylene))bis(ethan-1-one).

10. The method of manufacture of claim 8 in which the resulting compound is 4-(8-(4-acetylphenyl)-2-phenylimidazo[1,2-a]pyridin-6-yl)benzonitrile.

11. A method of treatment of a pathology or disease resulting from abnormal regulation of subtype 1A aldehyde dehydrogenase activity in a subject suffering therefrom, the method comprising administering a therapeutically effective amount of an inhibitor of aldehyde dehydrogenase proteins.

12. The method of treatment of claim 11 in which the inhibitor is a compound of Formula I, or a pharmaceutically acceptable salt or prodrug thereof: 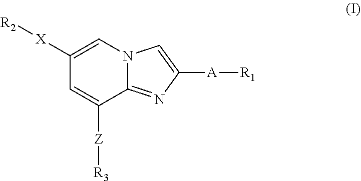
where A, X, and Z are each independently selected from aryl, heteroaryl, heterocyclic, or cyclic groups;
R₁, R₂, R₃ are each independently selected from hydrogen, hydroxy, amino, halogen, trifluoromethyl, cyano, nitro, carboxy, formyl, carbamoyl, acetyl, sulfamoyl, mercapto, acrylaldehyde, acrylate, acrylamide, acrylonitrile, dichloroacetamide, C1-C8 alkyl, C2-C8 alkenyl, C2-C8 alkynyl, C1-C6 alkoxy, C2-C6alkenyloxy, C2-C6alkynyloxy, C3-C7cycloalkyl, C3-C7heterocycloalkyl, hydroxyC1-C6alkyl, hydroxyC1-C6alkoxy, hydroxyC1-C6alkylamino, C1-C6alkoxyC1-C6alkyl, C1-C6alkoxyC1-C6alkylamino, aminoC1-C6alkyl, aminoC1-C6alkoxy, C1-C6alkylamino, di-C1-C6alkylamino, C1-C6-alkyldiamino, C1-C6alkoxycarbonyl, C1-C6alkoxyaminocarbonyl, C1-C6alkylthio, phosphateC1-C6alkylthio, sulfateC1-C6alkylthio, thioC1-C6alkyl, thioC1-C6alkoxy, thioC1-C6alkylamino, heterocycloC1-C6alkyl, heterocycloC1-C6alkoxy, heterocycloC1-C6alkylamino, phosphateC1-C6alkylamino, sulfateC1-C6alkylamino, C1-C6alkylsulfinyl, C1-C6alkylsulfonyl, C1-C6alkylthiocarboxamide, C1-C6alkylcarboxamide, C1-C6 alkylthioureyl, C1-C6alkylureyl, C1-C6dialkylamino, C1-C6alkoxycarbonyl, C1-C6dialkylcarboxamide, C1-C6 dialkylsulfonamide, C1-C6dialkylthiocarboxamido, C1-C6haloalkyl, C1-C6haloalkoxy, C1-C6guanidinealkyl, C1-C6cyanoalkyl, arylC1-C6alkoxy, heteroarylC1-C6alkoxy, cyclicC1-C6alkoxy, phosphateC1-C6alkoxy, sulfateC1-C6alkoxy, arylC1-C6alkylamino, heteroarylC1-C6alkylamino, cyclicC1-C6alkylamino, aryl, heteroaryl, heterocyclic, and cyclic groups;
and in which extant aryl, heteroaryl, heterocyclic, and cyclic groups are further functionalized with one or more substituents selected from hydrogen, halogen, trifluoromethyl, cyano, nitro, amino, hydroxy, carboxy, formyl, carbamoyl, sulfamoyl, mercapto, acrylaldehyde, acrylate, acrylamide, acrylonitrile, dichloroacetamide, C1-C6 alkyl, C2-C8 alkenyl, C2-C8 alkynyl, C1-C6 alkoxy, C2-C6 alkenyloxy, C2-C6 alkynyloxy, C1-C6 alkylcarboamino, C1-C6 alkylthio, C1-C6 alkylsulfonyl, C1-C6 alkylsulfonyl, C1-C6 alkylamino, di-C1-C6 alkylamino, amino C1-C6 alkyl, C3-C7 cycloalkyl, C1-C6 alkylcarbonyl, C2-C6 alkenylcarbonyl, C2-C6 alkynylcarbonyl, C1-C6 alkylaminocarbonyl, C1-C6 dialkyl aminocarbonyl, C1-C6 alkylsulfonyl aminocarbonyl, C1-C6 alkoxycarbonyl, arylaminocarbonyl and C3-C7 cycloalkylaminocarbonyl.

13. The method of treatment of claim 12 in which the compound is 1,1′-((2-phenylimidazo[1,2-a]pyridine-6,8-diyl)bis(4,1-phenylene))bis(ethan-1-one).

14. The method of treatment of claim 12 in which the compound is 4-(8-(4-acetylphenyl)-2-phenylimidazo[1,2-a]pyridin-6-yl)benzonitrile.

15. The method of treatment of claim 11 in which the pathology or disease is cancer.

16. The method of treatment of claim 11 in which the pathology or disease is inflammatory disease.

17. The method of treatment of claim 11 in which the pathology or disease is a tumor selected from head and neck, thyroid, lung, pleura, gastrointestinal tract, genitourinary tract, gynecological, breast, bone and soft tissue, skin, central nervous system, neuroblastoma, glioblastoma, retinoblastoma, rhabdomyosarcoma, hepatocellular carcinoma, glioblastoma multiformae, melanoma, squamous cell carcinoma, pancreatic cancer, mesothelioma, and rare pediatric tumor types.

18. The method of treatment of claim 11 in which the pathology or disease is an inflammatory disease exhibited within a body region selected from respiratory tree, lungs, gastrointestinal tract, bile duct, and the gallbladder.

19. The method of treatment of claim 11 in which the pathology or disease is selected from diabetes, psoriasis, rheumatoid arthritis, acute nephropathies, chronic nephropathies, arterial restenosis, auto-immune diseases, acute infections, eye diseases resulting from alteration of the microcirculation, and endometriosis.

20. A method of prevention of a pathology or disease resulting from abnormal regulation of subtype 1A aldehyde dehydrogenase activity in a subject suffering therefrom, the method comprising administering a prophylactically effective amount of an inhibitor of aldehyde dehydrogenase proteins.
